(19) United States
(12) Patent Application Publication
(10) Pub. No.: US 2025/0316378 A1
(43) Pub. Date: Oct. 9, 2025

(54) LIQUID BIOPSY FOR DIAGNOSIS OF EARLY OSTEOARTHRITIS

(71) Applicant: The Board of Trustees of the Leland Stanford Junior University, Stanford, CA (US)

(72) Inventors: Nidhi Bhutani, Redwood City, CA (US); Neety Sahu, Redwood City, CA (US)

(21) Appl. No.: 18/865,289

(22) PCT Filed: May 16, 2023
(86) PCT No.: PCT/US2023/022370

§ 371 Date: Nov. 12, 2024

Related U.S. Application Data
(60) Provisional application No. 63/342,873, filed on May 17, 2022.

(51) Int. Cl. G16H 50/20; G01N 15/1429; G01N 33/569; G16H 10/20

(52) U.S. Cl. CPC G16H 50/20; G01N 15/1429; G01N 33/56972; G16H 10/20; G01N 2333/70503; G01N 2333/70514; G01N 2333/70578; G01N 2333/70596; G01N 2333/7158

(57) ABSTRACT

Compositions and methods are provided for determining the presence of early-stage osteoarthritis (OA) in an individual by single cell profiling of a blood sample. Through use of machine learning, it is shown that immune cell features associated with OA are present and detectable in the early stages of OA and can be utilized for early detection of the disease.

### CROSS-REFERENCE

This application claims benefit of U.S. Provisional Patent Application No. 63/342,873, filed May 17, 2022, which application is incorporated herein by reference in its entirety.

### GOVERNMENT RIGHTS

This invention was made with Government support under contracts AR070864 and AR077530 awarded by the National Institutes of Health. The Government has certain rights in the invention.

### BACKGROUND

Osteoarthritis (OA) is an age-associated, chronic disease that affects 1 in every 5 adults above the age of 60, leading to joint dysfunction and persistent adverse effect on the quality of life. No disease-modifying drugs are available for OA, hence the clinical options are limited to pain management until the eventual surgery for total joint replacement. Although OA pathophysiology is distinct from autoimmune disease rheumatoid arthritis (RA), significant trafficking of immune cells is evident in OA joints. However, details regarding the immune cell types involved, the tissues that are infiltrated in the joint, the timing of infiltration, and the cellular cross-talk that results in the breakdown of joint homeostasis are still emerging. The advent of high-resolution single-cell techniques has made it possible to obtain precise maps of the immune landscape in patient tissues wherein rare and transitional cell types can be identified such that some of these outstanding questions can be answered.

However, assaying patients' tissues like the cartilage and synovium in the joint is difficult because procurement procedures are invasive and can even be detrimental to tissues like cartilage. Therefore, joint tissues harvested from the surgical wastes of patients undergoing total joint replacement surgeries, often at advanced stages of OA, are generally used to study the disease. Studying the peripheral blood can provide a systemic snapshot of the immune landscape that is readily accessible and non-invasive. Immune cell studies of the OA blood have the additional advantage of allowing larger cohort designs and serial monitoring from early to the late stages of the disease. Besides an increased understanding of OA pathogenesis, such studies can also help in earlier detection and patient stratification strategies, thereby contributing to precision medicine approaches to prevent and treat OA.

### SUMMARY

Compositions and methods are provided for determining the presence of early-stage osteoarthritis (OA) in an individual by single cell profiling of a blood sample. Through use of machine learning, it is shown that immune cell features associated with OA are present and detectable in the early stages of OA and can be utilized for early detection of the disease. The individual may be asymptomatic for OA, or may have joint abnormalities detectable only by imaging modalities. In some embodiments the individual is a human. In some embodiments the individual has a condition or injury that can pre-dispose to OA, e.g. a joint injury such as anterior cruciate ligament (ACL) tears, degenerative meniscal tears (DMT), etc.; genetic history; bone deformity; repetitive stress; metabolic disease, e.g. diabetes, hemochromatosis; and the like. Treatment of degenerative disease at a pre-clinical, sometimes asymptomatic, point in disease progression requires careful evaluation of the patient for early signs of disease. Treatment at this point is exceptionally valuable in that degeneration and loss of function is prevented.

In some embodiments, following immune cell profiling the individual is treated to ameliorate, diminish, actively treat, reverse or prevent injury, damage, or loss of articular cartilage or subchondral bone subsequent to the early stage of disease; and may prevent progression or reduce severity of OA. In some embodiments treatment is pharmacologic. In some embodiments treatment comprises physical and/or occupational therapy. In some embodiments treatment is surgical. In some embodiments treatment comprises clinical trial enrollment, where individuals can be stratified by likelihood of OA developing prior to the clinical trial.

It is shown here that immune cell populations that predict the presence of early stage OA include: (1) switched memory B CD27⁺IgD⁻CD24^{high} cells, (2) naïve B CD27⁻IgD⁺CXCR5⁺CD38⁺ cells; effector memory CD4 T cells with (3) CD27^{low}CD127^{high}CCR6⁺ and (4) CD27⁺CD127^{low}CCR6⁺, and (5) naïve CD4 T cells with CD27⁺CD127^{low}CXCR5⁺ phenotypes. The presence of altered levels of one or more, two or more, three or more, four, or five of the predictive cell populations in a patient sample comprising circulating immune cells, relative to a normal control, is diagnostic for the presence of OA. It will be understood by one of skill in the art that cells express multiple proteins on their surface, for example as shown in FIGS. 2 and 3, which can form the basis for phenotyping, and that these proteins provide a useful subset of the possible markers.

In some embodiments, a blood sample from an individual is contacted with a detectable agent, e.g. a detectably labeled antibody specific for a marker of interest, comprising an antibody for each of the disclosed markers in the OA-predictive populations. The set of markers is sufficient to phenotype at least one, at least two, at least three, at least four and may phenotype five or more OA-predictive immune cell populations. In an embodiment, the set of markers comprises: CD27; IgD; CD24; CD38; CXCR5; and may further comprise CD19. In an embodiment, the set of markers comprises: CD4; CD27; CD127; CCR6; and CXCR5. In an embodiment, the set of markers comprises: CD19; CD4; CD24; CD27; CD38; CD17; CXCR5; CCR6; and IgD.

The detectably labeled population is analyzed by flow cytometry at a single cell level. In some embodiments the flow cytometry is time-of-flight (TOF) mass cytometry. In some embodiments the flow cytometry is fluorescence activated flow cytometry. In some embodiments at least 10³ cells; at least 10⁴ cells; at least 10⁵ cells are analyzed. The resulting dataset is input into a predictive classification algorithm for a determination of whether the individual has early stage OA. The single cell data may be clustered into cell populations, e.g. by FlowSOM clustering. The algorithm may utilize a previously determined model for the prediction, e.g. a random forest model that has been trained on: samples from healthy individuals; individuals with ACL tear; individuals with DMT; etc.

The predictive analysis may further comprise analysis other than immune cell profiling, e.g. arthroscopy, radiographic imaging, ultrasound imaging, magnetic resonance imaging (MRI), computed tomography (CT), etc. The predictive analysis may further comprise determination of the presence of a molecule, e.g. C-reactive protein (CRP), a cytokine, antibody, cartilage component, protease, etc. or other clinical laboratory marker of inflammation, e.g. erythrocyte sedimentation rate (ESR), and compared to a control or reference value, wherein altered level of the molecular marker, in combination with the predictive cell classification, is indicative of early OA.

In one embodiment of the invention, the methods of determining the presence of early OA in an individual include obtaining a patient sample comprising circulating immune cells for analysis. Blood samples are a convenient source of circulating immune cells, particularly whole blood, although PBMC fractions also find use. The sample(s) is physically contacted with a panel of affinity reagents specific for markers that distinguish subsets of immune cells. Usually the affinity reagents comprise a detectable label, e.g. isotope, fluorophore, etc. Signal intensity of the markers is measured at a single cell level. The data is clustered and compared to measurements of the same from a training population. The data can be normalized for comparison.

Also described herein is a method for prediction of the presence of early OA, comprising: obtaining a dataset associated with an immune sample obtained from the subject, wherein the dataset comprises quantitative data from the markers disclosed herein; and analyzing the dataset classification with a predictive model, wherein a statistically significant match with a model disclosed herein is indicative of early OA. The data may be analyzed by a computer processor. The processor may be communicatively coupled to a storage memory for analyzing the data. The processor may be coupled to a flow cytometer, and may include algorithms for clustering cell populations, and predictive classification. Also described herein is a computer-readable storage medium storing computer-executable program code, the program code comprising: program code for storing and analyzing data obtained by the methods of the disclosure.

In other embodiments of the invention a device or kit is provided for the analysis of patient samples. Such devices or kits will include reagents that specifically identify one or more cells, indicative of the status of the patient, including without limitation affinity reagents. The reagents can be provided in isolated form, or pre-mixed as a cocktail suitable for the methods of the invention. A kit can include instructions for using the plurality of reagents to determine data from the sample; and instructions for statistically analyzing the data. The kits may be provided in combination with a system for analysis, e.g. a system implemented on a computer. Such a system may include a software component configured for analysis of data obtained by the methods of the invention.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1G. Circulating immune subpopulations are differentially abundant in OA. (a) Schematic representation of experimental design. Briefly, PBMCs from the whole blood of age-matched healthy donors and OA patients (n=12 each) were isolated and labeled with metal isotope-conjugated antibodies, followed by data acquisition by mass cytometry and analysis. (b) Manually gated major immune populations are visualized by tSNE plots in healthy and OA. (c) The abundance of manually gated immune cell types are represented as a percentage of CD45+CD66b− PBMCs. (d) tSNE visualization of significantly different subtypes of immune cells (manually gated) in healthy and OA. (e) Dot plot represents the abundance of all significantly different immune cell subtypes in all healthy and OA samples. (f) Proportion of all significantly different immune subtypes in healthy (inner circle) and OA (outer circle) cohorts. (g) Individual plots of immune subtypes found to be significantly different between healthy and OA. Data represents mean±standard deviation. Statistical significance was calculated by non-parametric Quade test to control for age and represented by p values after comparisons by Wilcoxon test and testing for multiple hypotheses by Bonferroni method.

FIGS. 2A-2H. OA landscape of the B cell repertoire defined by FlowSOM. (a) tSNE representation of significantly different clusters of B cells in healthy and OA cohorts (n=12 each), identified by unsupervised hierarchical clustering by FlowSOM. (b) Heatmap represents medians of markers (z-scored column-wise) identified as memory and naïve subtypes of B cells that were differentially abundant in OA. Percent abundance of significantly different (c) naïve B cell clusters, (d) switched memory B cell metacluster (e) switched memory B cell clusters in OA. (f) tSNE map of significantly different monocyte clusters. (g) Heatmap represents the median of markers used to identify the monocyte clusters. (h) Half boxplots of significantly different monocyte clusters in OA when compared to the healthy cohort. Dots in the half boxplots represent the cluster abundance of individual samples. Line inside the boxplot is the median at 50^{th} percentile, and upper and lower whiskers denote data within 1.5 times the interquartile range under 75^{th} and 25^{th} percentiles respectively. Statistical significance between healthy and OA clusters was calculated by non-parametric Quade test to control for age and represented by p values after comparisons by Wilcoxon test and adjusting for multiple hypotheses by Bonferroni test.

FIGS. 3A-31. T cell repertoire in OA as defined by FlowSOM. (a) tSNE projection of significantly different CD4T cell clusters in healthy and OA cohorts (n=12 each). (b) Heatmap represents the medians of markers used for the identification of the FlowSOM defined clusters. The frequency of significantly different (c) naïve (d) central memory (e) terminal effector CD4T cell clusters in OA is represented as a percentage of total CD4T cells in half boxplots. (f) tSNE projection of significantly different CD8T clusters in healthy and OA (n=12 each). (g) Heatmap of marker medians used to identify the CD8T clusters. Half boxplots of significantly different (h) effector memory (EM) and (i) terminal effector CD8 T cell clusters in OA when compared to healthy. Dots in the half boxplots represent the distribution of individual samples in a cohort. Line inside the boxplot is the median at 50^{th} percentile, and upper and lower whiskers denote data within 1.5 times the interquartile range under 75^{th} and 25^{th} percentiles respectively. Statistical significance between healthy and OA clusters was calculated by non-parametric quade test to control for age and represented by p values after comparisons by Wilcoxon test and adjusting for multiple hypotheses by Bonferroni test.

FIGS. 4A-4C. Circulating immune cell profile in OA is distinct from healthy. (a) Sample-wise frequency distribution of all significantly different immune cell clusters identified by FlowSOM is represented as the percent of each cell type per row. (b) Heatmap of cluster frequencies of the significantly different clusters in all healthy and OA samples. Dendrograms result from unsupervised clustering where distance is measured by cosine similarity. (c) Principal component analysis (PCA) of the immune profiles comprising the significantly different clusters between healthy and OA.

FIGS. 5A-5M. Circulating immune cell landscape in DMT. (a) tSNE projections of immune cell subtypes (defined by manual gating) that were significantly different between healthy and OA, and also significantly different in DMT when compared to healthy. (b) The frequency of Th2 and Th14 subsets of CD4T cells are represented as a percentage of total CD45+CD66b− cells in healthy, OA and DMT cohorts (n=12 each). Data represent mean±standard deviation. Significance is calculated by Quade test to control for an age followed by post-hoc Dunn's test with Bonferroni correction. Subsequent analyses were conducted on immune subpopulations defined by hierarchical clustering by FlowSOM, as follows: (c) tSNE projections of differentially abundant FlowSOM-defined B cell clusters in healthy, OA and DMT cohorts. Half boxplots representing cluster frequencies as a percentage of total B cells in (d) naïve (e) switched memory B subsets. Clusters were significantly different in DMT when compared with healthy, as they were in OA. (f) tSNE projection of monocyte cluster significantly different in both OA and DMT when compared with healthy (n=12 each). (g) Half boxplot of the frequency of the monocyte cluster identified as CD14low classical monocyte as a percentage of total monocytes analyzed. (h) tSNE projections (n=12 per group) and (i) half boxplots of frequencies of naïve CD4T cell clusters that were significantly different in DMT when compared to healthy, as they were in OA (except where indicated as ns, not significant). Dots in the half boxplots represent distribution of individual samples in a cohort. Line inside the boxplot is the median at 50^{th} percentile, and upper and lower whiskers denote data within 1.5 times the interquartile range under 75^{th} and 25^{th} percentiles respectively. Statistical significance between healthy, OA and DMT clusters was calculated by non-parametric quade test to control for age and represented by p values after comparisons by Dunn's test and adjusting for multiple hypotheses by Bonferroni test. Sample-wise frequency distribution of identified clusters between (j) healthy and DMT, and (k) OA and DMT is shown as percentage of cell type per row in dot plots. (l) Circular plots represent proportional distribution of significant clusters in heathy, OA and DMT cohorts. (m) PCA plot depicts spatial distribution of each sample based on their cluster frequencies.

FIGS. 6A-6N. Immune cell landscape in ACL. (a) tSNE projections of differentially abundant clusters between healthy, OA, and ACL cohorts (n=12 each) as defined by FlowSOM. Frequencies of significantly different (b) naïve B and (c) memory B cell clusters are represented as a percentage of total B cells. (d) tSNE projections and (e) frequencies of differentially abundant monocyte clusters in healthy, OA and ACL clusters (n=12 each). tSNE representation of differentially abundant (f) CD4T cell and (h) CD8T cell clusters between healthy, OA and ACL cohorts (n=12 each). Half boxplots representing frequencies of significantly different (g) CD4T (i, j) CD8T cell clusters between healthy, OA and ACL cohorts as percentages of total CD4T and CD8T cells respectively. Dots in the half boxplots represent the distribution of individual samples in a cohort. Line inside the boxplot is the median at 50^{th} percentile, and upper and lower whiskers denote data within 1.5 times the interquartile range under 75^{th} and 25^{th} percentiles respectively. Statistical significance between healthy, OA, and ACL clusters was calculated by non-parametric Quade test to control for age and represented by p values after comparisons by Dunn's test and adjusting for multiple hypotheses by Bonferroni test. Sample-wise frequency distribution of identified clusters between (k) healthy and ACL, and (l) OA and ACL is represented as a percentage of cell type per row in dot plots. (m) Circular plots represent the proportional distribution of significant clusters in heathy, OA and ACL cohorts. (n) PCA plot depicts the spatial distribution of each sample based on their cluster frequencies.

FIG. 7A-7G. Feature selection and disease classification by machine learning. (a) Schematic outlines the design of datasets for selection and identification of most OA-like immune profiles in DMT and ACL samples. Briefly, a training dataset comprising FlowSOM-defined immune profiles of age-matched healthy (n=12) and OA (n=12) groups along with young healthy (n=7) was compiled and the only clusters that were significantly different between age-matched healthy and OA were considered for feature selection by Boruta. Selected features in the training dataset were then used to train the random forest algorithm to identify and predict OA-like profiles in a test dataset. The test dataset consisted of immune profiles of selected features in DMT (n=11), ACL (n=11), and a validation cohort of young healthy (n=5) profiles unseen by the algorithm. FlowSOM defined immune cell clusters belonging to (b) B cells and (c) CD4T cells were significantly different between age-matched healthy and OA and are represented by half boxplots. Statistical significance between healthy, OA, and ACL clusters was calculated by non-parametric quade test to control for age and represented by p values after comparisons by Dunn's test and adjusting for multiple hypotheses by Bonferroni test. (d) Among the statistically significant clusters, the clusters/features in green were deemed important by the Boruta algorithm. (e, f) Heatmap represents the median of markers used to identify the important features (g) Heatmap represents the percent probability of showing a healthy or OA-like profile in the test dataset of unseen young healthy, DMT and ACL cohorts predicted by random forest classifier. The Red line in the barplot annotation of percent probability of OA-like profile in samples of the test cohort denotes a self-determined cutoff at 70%.

FIG. 8. Manual gating strategy for the identification of immune cells. Cells were first gated on the expression of DNA (labeled with Ir191) and cisplatin to identify live cells as positive for DNA and negative for cisplatin. Cells were gated as CD45⁺CD66b⁻ on live cells were the parent population to identify major immune cells and their subtypes, namely monocytes (classical and non-classical), B cells (plasmoblasts, naïve, transitional, memory resting and switched memory), NK cells (CD16⁺ and CD16⁻), dendritic cells (mDCs and pDCs), CD8 T cells (activated, naïve, central memory, effector memory and terminal effector) and CD4 T cells (activated, naïve, central memory, effector memory and terminal effector, Th1, Th2, Th17, activated CCR4⁺ Tregs, CCR4⁺ memory Treg and CCR4⁺ naïve Tregs).

FIG. 9. PCA biplot of variables. PCA biplot depicts the PCA scores and loading variables (clusters) for all samples. Lengths of the lines indicate the magnitude of influence of each variable or cluster.

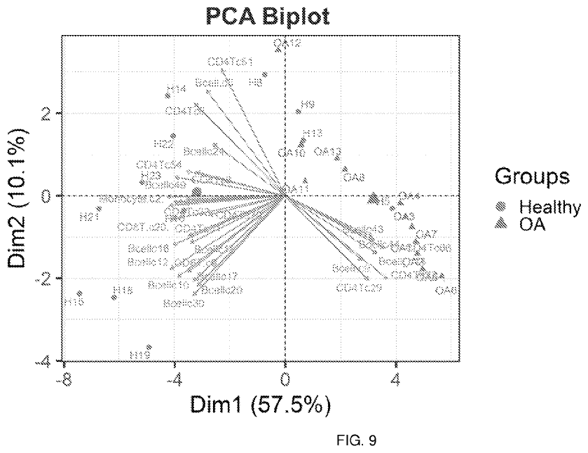

FIG. 10. Error rate graph of the random forest model. Graph represents error rate per number of trees for random forest model. A total of 1000 trees were utilized in the final model. The out-of-bag (OOB) error is represented in black, error for healthy in red and OA in green for the training dataset.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Osteoarthritis (OA). OA affects nearly 27 million people in the United States, accounting for 25% of visits to primary care physicians, and half of all prescriptions for non-steroidal anti-inflammatory drugs (NSAIDs). It is a chronic arthropathy characterized by disruption and potential loss of joint cartilage along with other joint changes, including bone remodeling such as bone hypertrophy (osteophyte formation), subchondral sclerosis, and formation of subchondral cysts. OA results in the degradation of joints, including degradation of articular cartilage and subchondral bone, resulting in mechanical abnormalities and joint dysfunction. Symptoms may include joint pain, tenderness, stiffness, sometimes an effusion, and impaired joint function. A variety of causes can initiate processes leading to loss of cartilage in OA.

OA may begin with joint damage caused by trauma to the joint; mechanical injury to the meniscus, articular cartilage, a joint ligament, or other joint structure; defects in cartilage matrix components; and the like. Mechanical stress on joints may underlie the development of OA in many individuals, with the sources of such mechanical stress being many and varied, including misalignment of bones as a result of congenital or pathogenic causes; mechanical injury; overweight; loss of strength in muscles supporting joints; and impairment of peripheral nerves, leading to sudden or dys-coordinated movements that overstress joints.

In synovial joints there are at least two movable bony surfaces that are surrounded by the synovial membrane, which secretes synovial fluid, a transparent alkaline viscid fluid that fills the joint cavity, and articular cartilage, which is interposed between the articulating bony surfaces. The earliest gross pathologic finding in OA is softening of the articular cartilage in habitually loaded areas of the joint surface. This softening or swelling of the articular cartilage is frequently accompanied by loss of proteoglycans from the cartilage matrix. As OA progresses, the integrity of the cartilage surface is lost and the articular cartilage thins, with vertical clefts extending into the depth of the cartilage in a process called fibrillation. Joint motion may cause fibrillated cartilage to shed segments and thereby expose the bone underneath (subchondral bone). In OA, the subchondral bone is remodeled, featuring subchondral sclerosis, subchondral cycts, and ectopic bone comprising osteophytes. The osteophytes (bone spurs) form at the joint margins, and the subchondral cysts may be filled with synovial fluid. The remodeling of subchondral bone increases the mechanical strain and stresses on both the overlying articular cartilage and the subchondral bone, leading to further damage of both the cartilage and subchondral bone.

The tissue damage stimulates chondrocytes to attempt repair by increasing their production of proteoglycans and collagen. However, efforts at repair also stimulate the enzymes that degrade cartilage, as well as inflammatory cytokines, which are normally present in only small amounts. Inflammatory mediators trigger an inflammatory cycle that further stimulates the chondrocytes and synovial lining cells, eventually breaking down the cartilage. Chondrocytes undergo programmed cell death (apoptosis) in OA joints.

OA should be suspected in patients with gradual onset of joint symptoms and signs, particularly in older adults, usually beginning with one or a few joints. Pain can be the earliest symptom, sometimes described as a deep ache. Pain is usually worsened by weight bearing and relieved by rest but can eventually become constant. Joint stiffness in OA is associated with awakening or inactivity. If OA is suspected, plain x-rays should be taken of the most symptomatic joints. X-rays generally reveal marginal osteophytes, narrowing of the joint space, increased density of the subchondral bone, subchondral cyst formation, bony remodeling, and joint effusions. Standing x-rays of knees are more sensitive in detecting joint-space narrowing. Magnetic resonance imaging (MRI) can be used to detect cartilage degeneration, and several MRI-based based scoring systems exist for characterizing the severity of OA (Hunter et al, PM R. 2012 May; 4(5 Suppl):S68-74).

OA commonly affects the hands, feet, spine, and the large weight-bearing joints, such as the hips and knees, although in theory any joint in the body can be affected. As OA progresses, the affected joints appear larger, are stiff and painful, and usually feel better with gentle use but worse with excessive or prolonged use. Treatment generally involves a combination of exercise, lifestyle modification, and analgesics. If pain becomes debilitating, joint-replacement surgery may be used to improve quality of life.

In addition to affecting humans, OA and joint degeneration also frequently impacts animals, including dogs, cats, horses, and other animals in which it can causes significant joint pain and dysfunction. Osteoarthritis (OA) is the most common form of arthritis in dogs, affecting approximately a quarter of the population. It is a chronic joint disease characterized by loss of joint cartilage, thickening of the joint capsule and new bone formation around the joint (osteophytosis) and ultimately leading to pain and limb dysfunction. The majority of OA in dogs occur secondarily to developmental orthopedic disease, such as cranial cruciate ligament disease, hip dysplasia, elbow dysplasia, OCD, patella (knee cap) dislocation. In a small subset of dogs, OA occurs with no obvious primary causes and can be related to genetic and age. Other contributing factors to OA in dogs include body weight, obesity, gender, exercise, and diet.

Treatment of OA includes, for example, pharmacologic treatment such as doxycycline, bisphosphonates, and licofelone. Other OA drugs and targets include, for example, inhibition of cartilage matrix degradation with MMP-inhibitor PG-116800; Cartilage matrix regeneration with Sprifermin, BMP-7, or OP-1; bisphosphonates; bone turnover with Zoledronic acid, Risedronate; AXS-02 (disodium zoledronate tetrahydrate); inhibition of bone degradation with Cathepsin K inhibitor MIV-711; inhibition of IL-1 with Anakinra (IL-1 receptor antagonist), AMG 108 (fully human monoclonal antibody to IL-1R1), Lutikizumab (anti IL-1 a/p antibody); anti-tumor necrosis factor-alpha, e.g. Adalimumab, Etanercept, Infliximab; Hydroxychloroquine; inhibition of I-κB kinase with SAR113945 (I-kB kinase inhibitor); inhibition of p38 MAP kinase with FX-005; agents that act on Cox-2, e.g. metformin; agents that act on HMG-CoA reductase, e.g. simvastatin, atorvastatin, Fluvastatin, lovastatin, nystatin, pravastatin, rosuvastatin, and the like. Anti-inflammatories such as NSAIDs, opiates, intra-articular corticosteroids, and hyaluronic acid derivatives injected into the joint are also used.

Early osteoarthritis. Assessment of OA may use the Kellgren Lawrence (KL) grading system (Kellgren and Lawrence, Ann. Rheum. Dis., 16:494-502, 1957, herein specifically incorporated by reference). The KL grading system relies on an anterior-posterior (AP) radiograph and is as follows: grade 0=no features of OA; grade 1=presence of OA is doubtful, presence of minute osteophyte(s), unchanged joint space; grade 2=minimal OA, definite osteophyte(s), unchanged joint space; grade 3=moderate OA, moderate diminution of joint space; grade 4=severe OA, joint space greatly reduced with sclerosis of subchondral bone. For the purposes of the present invention, the KL score is less than 3, desirably less than 2, and in some embodiments is less than one. In some embodiments, the presence of early stages of arthritis is indicated by lack of definite joint space narrowing, lack of osteophytes (Kellgren-Lawrence Grade <2) but with positive results in at least one imaging marker, e.g. from an examination of one or more joints using noninvasive procedures including radiographic imaging and MRI for features including, for example, cartilage breakdown, decreased synovial space, and the like.

Individuals with pre-clinical or early-OA are those at increased risk of developing OA, as evidenced by biochemical, imaging, or clinical markers. Conditions or events that predispose to the development of OA include, without limitation, a history of injury to a joint; clinically or radiographically diagnosed meniscal injury with or without surgical intervention; a ligamentous sprain with clinically or radiographically diagnosed anterior or posterior cruciate or medial or lateral collateral ligament injury (Chu et al, Arthritis Res Ther. 2012 14(3):212. PMID: 22682469); clinically measured limb-length discrepancy; obesity with a current, or prolonged historical period of, BMI >27; or biomechanical features of abnormal gait or joint movement. In general, a determination of pre-clinical OA is associated with one or more, two or more, three or more parameters of joint pathology including, without limitation and relative to a healthy control sample, cartilage proteoglycan loss; cartilage damage; or elevated levels of degradative enzymes, the presence of products of cartilage or extracellular matrix degradation or bone remodeling. Humans at risk for OA, who have pre-OA, and who have early-stage OA are often asymptomatic, but a subset of patients experience joint pain due to cartilage injury (e.g. meniscal injury), ligamentous injury (e.g. tearing of the anterior cruciate ligament), or another joint abnormality.

MRI-detected imaging markers indicative of the presence of early or pre-clinical OA include cartilage edema, cartilage proteoglycan loss, cartilage matrix loss, bone marrow edema, articular cartilage fissures, articular cartilage degeneration, a meniscal tear, an anterior cruciate ligament tear, a posterior cruciate ligament tear, and other abnormalities of the cartilage or ligaments in the joint. Ultrasound will show evidence of cartilage edema or damage. Arthroscopy can allow direct detection or visualization of cartilage edema, cartilage softening, cartilage thinning, cartilage fissures, cartilage erosion, or other cartilage abnormalities. Cartilage damage is frequently defined by the Outerbridge classification criteria or similar directly observed changes within the joint. For example, one such scoring system defines the presence of damage is as follows: grade 0=normal cartilage; grade I: softening and swelling of cartilage; grade II: a partial-thickness defect in the cartilage with fissures on the surface that do not reach subchondral bone or exceed 1.5 cm in diameter; grade III: fissures in the cartilage that extend to the level of subchondral bone in an area with a diameter of more than 1.5 cm. Humans at risk for OA or with “pre-clinical OA” may be asymptomatic but may have signs of cartilage damage, meniscal damage, ligament damage, or other abnormalities of the joint.

Mass cytometry. Elemental mass spectrometry-based flow cytometry (mass cytometry) is a method to characterize single cells or particles with elemental metal isotope-labeled binding reagents. Because there are many stable metal isotopes available, and little overlap between measurement channels, dozens of molecules (parameters) can be readily measured. An example of a mass cytometer used to read the metal tags is an inductively-coupled plasma mass spectrometer (ICP-MS). In a typical workflow (similar to fluorescence based cytometry), cells are first incubated with antibodies/affinity binders conjugated to pure isotopes and subsequently the cell suspension is injected as a single cell stream into the mass cytometer. Single cell droplets are generated via nebulization and are carried by an argon gas stream into about 7500 degrees Kelvin plasma where each single cell is completely atomized and ionized. Thereby generated metal ions are then directed into a time-of-flight (TOF) mass spectrometer and the mass over charge ratio and number of metal ions is measured per cell and thereby the abundance of the target epitope/molecules.

As used herein, the term “elemental analysis” refers to a method by which the presence and/or abundance of elements of a sample are evaluated. “Capacitively coupled plasma” (CCP) means a source of ionization in which a plasma is established by capacitive coupling of radiofrequency energy at atmospheric pressure or at a reduced pressure (typically between 1 and 500 Torr) in a graphite or quartz tube.

“Mass spectrometer” means an instrument for producing ions in a gas and analyzing them according to their mass/charge ratio. “Microwave induced plasma” (MIP) means a source of atomization and ionization in which a plasma is established in an inert gas (typically nitrogen, argon or helium) by the coupling of microwave energy. The frequency of excitation force is in the GHz range. “Glow discharge” (GD) means a source of ionization in which a discharge is established in a low pressure gas (typically between 0.01 and 10 Torr), typically argon, nitrogen or air, by a direct current (or less commonly radiofrequency) potential between electrodes. “Graphite furnace” means a spectrometer system that includes a vaporization and atomization source comprised of a heated graphite tube. Spectroscopic detection of elements within the furnace may be performed by optical absorption or emission, or the sample may be transported from the furnace to a plasma source (e.g. inductively coupled plasma) for excitation and determination by optical or mass spectrometry.

In some embodiments the methods utilize ICP-MS. In some embodiments the ICP-MS is performed with solution analysis, for example ELAN DRC II, Perkin-Elmer. In other embodiments the analysis is performed with a mass cytometer (e.g. CyTOF, DVS Sciences), which uses a nebulizer to administer a suspension of cells, beads, or other particles in a single-particle stream to an ICP-MS chamber, thereby yielding single particle/cell data similar to a flow cytometer. Alternatively the analysis is performed by an elemental analysis-driven imaging system (e.g. laser ablation ICP-MS). Devices for such analytic methods are known in the art.

The term “flow cytometry” as used herein refers to a method and a process whereby cells within a sample can be detected and identified when transversing past a detector within an apparatus containing a detecting source and a flowing apparatus, e.g. FACS and mass cytometry. Flow cytometry can provide an alternative analysis means, rather than mass cytometry.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of arthritis in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of osteoarthritis. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

A “sample” in the context of the present teachings refers to any biological sample that is isolated from a subject, generally a blood sample, which may comprise circulating immune cells. “Blood sample” can refer to whole blood or a fraction thereof, including blood cells, plasma, white blood cells or leucocytes. Samples can be obtained from a subject by means including but not limited to venipuncture, biopsy, needle aspirate, lavage, scraping, surgical incision, or intervention or other means known in the art.

The term also encompasses samples that have been manipulated in any way after their procurement, such as by treatment with reagents; washed; or enrichment for certain cell populations.

Cells for use in the methods as described above may be collected from a sample from a subject or a donor, and may optionally may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The collected and optionally enriched cell population may be used immediately or may be frozen at liquid nitrogen temperatures and stored, being thawed and capable of being reused. The cells will usually be stored in 10% DMSO, 50% FCS, 40% RPMI 1640 medium.

A “dataset” is a set of numerical values resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring antibody binding, or other methods of quantitating a signaling response. The phrase also encompasses receiving a set of data, e.g., from a third party that has processed the sample to experimentally determine the dataset.

“Measuring” or “measurement” in the context of the present teachings refers to determining the presence, absence, quantity, amount, or effective amount of a cell population in a clinical or subject-derived sample, including the presence, absence, or concentration levels of such cells, and/or evaluating the values or categorization of a subject's clinical parameters based on a control, e.g. baseline levels of the cell population.

Classification can be made according to predictive modeling methods that set a threshold for determining the probability that a sample belongs to a given class. The probability preferably is at least 50%, or at least 60% or at least 70% or at least 80% or higher. Classifications also can be made by determining whether a comparison between an obtained dataset and a reference dataset yields a statistically significant difference, where the reference dataset may correspond to the results for a healthy control cell population. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

Classification is the process of recognizing, understanding, and grouping ideas and objects into preset categories or “sub-populations.” Using pre-categorized training datasets, machine learning programs use a variety of algorithms to classify future datasets into categories. Classification algorithms in machine learning use input training data to predict the likelihood that subsequent data will fall into one of the predetermined categories. An analytic classification process may use any one of a variety of statistical analytic methods to manipulate the quantitative data and provide for classification of the sample. Examples of useful methods include linear discriminant analysis, recursive feature elimination, a prediction analysis of microarray, a logistic regression, a CART algorithm, a FlexTree algorithm, a LART algorithm, a random forest algorithm, a MART algorithm, machine learning algorithms; etc. Using any one of these methods, a protein distribution pattern may be used to generate a predictive model. In the generation of such a model, a dataset comprising control, and OA are used as a training set. A training set will contain data for one or more different distributions of interest. In some embodiments a decision tree is used to order classes on a precise level, for example with a random forest algorithm.

The predictive ability of a model can be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold can refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity can be at least about at least about 0.7, at least about 0.75, at least about 0.8, at least about 0.85, at least about 0.9, or higher.

The raw data may be initially analyzed by measuring the values for each marker, usually in triplicate or in multiple triplicates; and the cells may be clustered into populations, e.g. with flowSOM. The data may be manipulated, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc., Series B, 26:211-246), etc. The data are then input into a predictive model, which will classify the sample according to the state. The resulting information may be transmitted to a patient or health professional.

The term “specific binding member” as used herein refers to a member of a specific binding pair, i.e. two molecules, usually two different molecules, where one of the molecules through chemical or physical means specifically binds to the other molecule. For the purposes of the present invention, one of the molecules is an analyte as defined above, and generally the specific binding member is labeled for detection of fluorescence or elemental analysis, as known in the art.

The complementary members of a specific binding pair are sometimes referred to as a ligand and receptor; or receptor and counter-receptor. Specific binding indicates that the agent can distinguish a target antigen, or epitope within it, from other non-target antigens. It is specific in the sense that it can be used to detect a target antigen above background noise (“non-specific binding”). For example, a specific binding partner can detect a specific sequence or a topological conformation. A specific sequence can be a defined order of amino acids or a defined chemical moiety (e.g., where an antibody recognizes a phosphotyrosine or a particular carbohydrate configuration, etc.) which occurs in the target antigen. The term “antigen” is issued broadly, to indicate any agent which elicits an immune response in the body. An antigen can have one or more epitopes.

Binding pairs of interest include antigen and antibody specific binding pairs, complementary nucleic acids, peptide-MHC-antigen complexes and T cell receptor pairs, biotin and avidin or streptavidin; carbohydrates and lectins; complementary nucleotide sequences; peptide ligands and receptor; effector and receptor molecules; hormones and hormone binding protein; enzyme cofactors and enzymes; enzyme inhibitors and enzymes; and the like. The specific binding pairs may include analogs, derivatives and fragments of the original specific binding member. For example, an antibody directed to a protein antigen may also recognize peptide fragments, chemically synthesized peptidomimetics, labeled protein, derivatized protein, etc. so long as an epitope is present.

Immunological specific binding pairs include antigens and antigen specific antibodies; and T cell antigen receptors, and their cognate MHC-peptide conjugates. Suitable antigens may be haptens, proteins, peptides, carbohydrates, etc. Recombinant DNA methods or peptide synthesis may be used to produce chimeric, truncated, or single chain analogs of either member of the binding pair, where chimeric proteins may provide mixture(s) or fragment(s) thereof, or a mixture of an antibody and other specific binding members. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

A nucleic acid based binding partner such as an oligonucleotide can be used to recognize and bind DNA or RNA based analytes. The term “polynucleotide” as used herein may refer to peptide nucleic acids, locked nucleic acids, modified nucleic acids, and the like as known in the art. The polynucleotide can be DNA, RNA, LNA or PNA, although it is not so limited. It can also be a combination of one or more of these elements and/or can comprise other nucleic acid mimics.

Binding partners can be primary or secondary. Primary binding partners are those bound to the analyte of interest. Secondary binding partners are those that bind to the primary binding partner.

In one embodiment analysis is performed on a mass cytometer, in which cells are introduced into a fluidic system and introduced into the mass cytometer one cell at a time. In one embodiment, cells are carried in a liquid suspension and sprayed into a plasma source by means of a nebulizer. In another embodiment, the cells may be hydrodynamically focused one cell at a time through a flow cell using a sheath fluid. In particular embodiments, the cells may be compartmentalized in the flow cell by introduction of an immiscible barrier, e.g., using a gas (e.g., air or nitrogen) or oil, such that the cell is physically separated from other cells that are passing through the flow cell. The cells may be compartmentalized prior to or during introduction of the cell into the flow cell by introducing an immiscible material (e.g., air or oil) into the flow path.

The general principles of mass cytometry, including methods by which single cell suspensions can be made, methods by which cells can be labeled, methods for atomizing particles and methods for performing elemental analysis on particles, as well as hardware that can be employed in mass cytometry, including flow cells, ionization chambers, reagents, mass spectrometers and computer control systems are known and are reviewed in a variety of publications including, but not limited to Bandura et al Analytical Chemistry 2009 81 6813-6822), Tanner et al (Pure Appl. Chem 2008 80: 2627-2641), U.S. Pat. No. 7,479,630 (Method and apparatus for flow cytometry linked with elemental analysis) and U.S. Pat. No. 7,135,296 (Elemental analysis of tagged biologically active materials); and published U.S. patent application 20080046194, for example, which publications are incorporated by reference herein for disclosure of those methods and hardware.

The results of such analysis may be compared to results obtained from reference compounds, concentration curves, controls, etc. The comparison of results is accomplished by the use of suitable deduction protocols, AI systems, statistical comparisons, etc.

In particular embodiments, the method described above may be employed in a multiplex assay in which a heterogeneous population of cells is labeled with a plurality of distinguishably labeled binding agents (e.g., a number of different antibodies). After the population of cells is labeled, the cells are introduced into the flow cell, and individually analyzed using the method described above, where the viable cells are distinguished from non-viable cells by the presence of platinum derived from the viability reagent.

The analyte distribution pattern may be generated from a cell sample using any convenient protocol. The readout may be a mean, average, median or the variance or other statistically or mathematically-derived value associated with the measurement. The readout information may be further refined by direct comparison with the corresponding reference or control pattern. A pattern may be evaluated on a number of points: to determine if there is a statistically significant change at any point in the data matrix; whether the change is an increase or decrease in prevalence of an isoform; and the like. The absolute values will display a variability that is inherent in live biological systems.

### Methods

Analysis of biological samples, e.g. blood-based samples, obtained from an individual is used to obtain a determination of changes in immune cell subsets, which are shown herein to be predictive of the presence of early OA.

The sample can be any suitable type that allows for the analysis of one or more cells, proteins and metabolites, preferably a blood sample. Samples can be obtained once or multiple times from an individual. The cells can be separated from body samples by red cell lysis, centrifugation, elutriation, density gradient separation, apheresis, affinity selection, panning, FACS, centrifugation with Hypaque, solid supports (magnetic beads, beads in columns, or other surfaces) with attached antibodies, etc.

A phenotypic profile of a population of cells is determined by measuring at a single cell level the presence of specific markers. It is understood that marker levels can exist as a distribution and that a marker used to classify a cell can be a particular point on the distribution but more typically can be a portion of the distribution. In some embodiments of the invention, different gating strategies can be used in order to analyze a specific cell population (e.g., only CD4⁺ T cells) in a sample of mixed cell population. These gating strategies can be based on the presence of one or more specific surface markers. The following gate can differentiate between dead cells and live cells and the subsequent gating of live cells classifies them into, e.g. myeloid blasts, monocytes and lymphocytes. A clear comparison can be carried out by using two-dimensional contour plot representations, two-dimensional dot plot representations, and/or histograms.

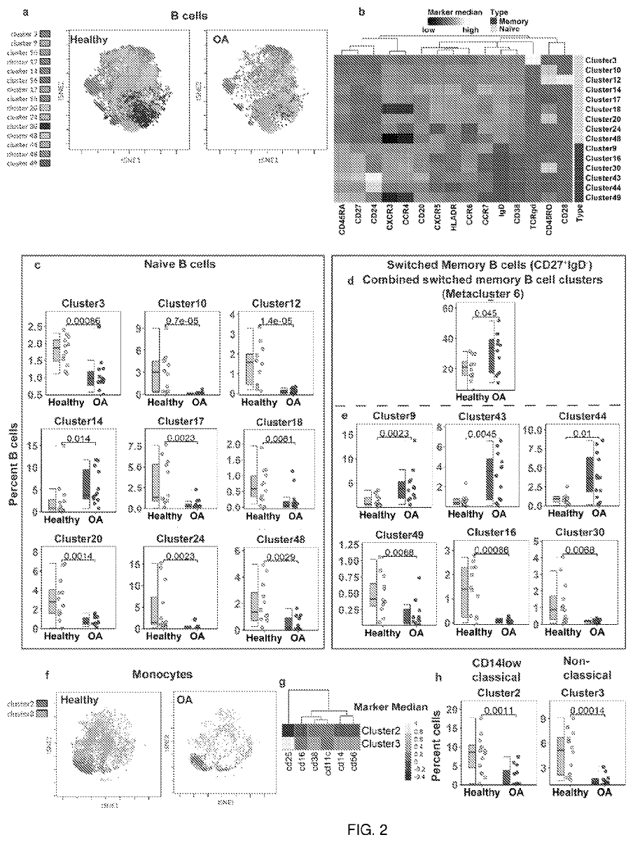

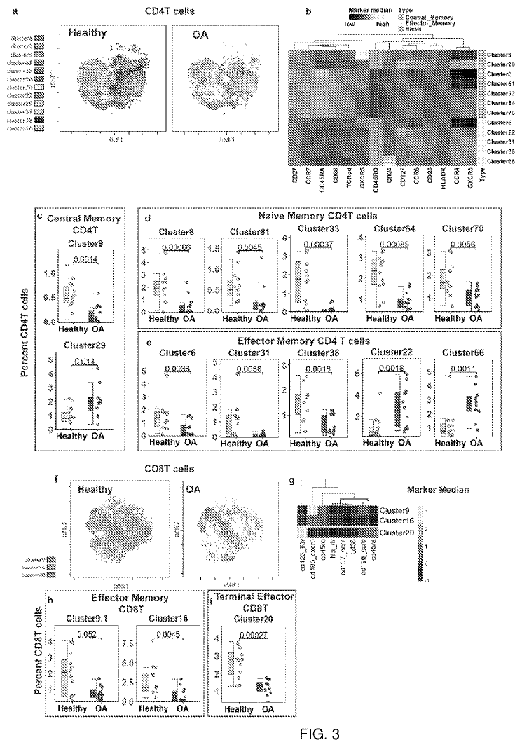

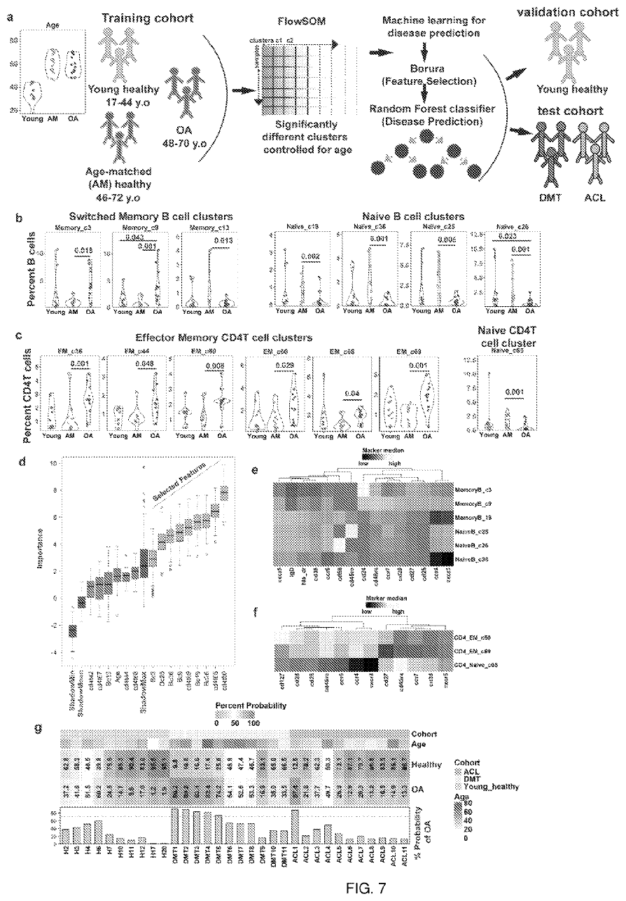

In some embodiments, the profiling measures the concentration of at least 1, at least 2, at least 3, at least 4, and may include all 5 of the immune cell populations (1) switched memory B CD27⁺IgD⁻CD24^{high} cells, (2) naïve B CD27⁺IgD⁺CXCR5⁺CD38⁺ cells; effector memory CD4 T cells with (3) CD27^{low}CD127^{high}CCR⁺ and (4) CD27⁺CD127^{low}CCR6⁺, and (5) naïve CD4 T cells with CD27⁺CD127^{low}CXCR5⁺. The data supporting the relevance of these cell populations in a predictive model.

| | Disease | Cluster no. | Cluster no. (Identified by |
|---|---|---|---|
| Cell phenotype | feature | (FlowSOM) | machine learning) |
| Switched memory B cell | Expanded | Corresponds to clusters 43 and | N/A |
| CD27⁺IgD⁻CD24^{high} | | 44 (FIG. 2B and 2D) | |
| Naïve B cell | Depleted | Corresponds to clusters 18, 20, | Clusters 19, 25, 26 and |
| IgD⁺CXCR5⁺CD38⁺ | | 17 and 48 (FIG. 2B and C). | 36 (FIG. 7B and E) |
| Effector memory CD4 T cell | Expanded | Corresponds to cluster 22 | Cluster 50 |
| CD27^{low}CD127^{high}CCR6⁺ | | (FIG. 3B and E) | (FIG. 7C and F) |
| Effector memory CD4 T cell | Expanded | Corresponds to cluster 66 | Cluster 69 |
| CD27⁺CD127^{low}CCR6⁺ | | (FIG. 3B and E | (FIG. 7C and F) |
| Naïve memory CD4T cells | Depleted | Corresponds to cluster 8 | Cluster 69 |
| (CD27⁺CD127^{low}CXCR5⁺) | | (FIG. 3B and D) | (FIG. 7C and F) |

A population that is “expanded” relative to a healthy control population may be increased at least about 1.25-fold, at least about 1.5-fold, at least about 1.75-fold, at least about 2-fold, at least about 2.5-fold, at least about 3-fold or more. A population that is depleted relative to a healthy control population may be decreased at least about 1.25-fold, at least about 1.5-fold, at least about 1.75-fold, at least about 2-fold, at least about 2.5-fold, at least about 3-fold or more.

Samples may be obtained at one or more time points. Where a sample at a single time point is used, comparison is made to a reference “base line” level for the feature, which may be obtained from a training set data as disclosed herein.

In some embodiment, the methods of the invention include the use of liquid handling components. The liquid handling systems can include robotic systems comprising any number of components. In addition, any or all of the steps outlined herein can be automated; thus, for example, the systems can be completely or partially automated. See U.S. Ser. No. 61/048,657. As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a mass cyometer; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this can be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

Individuals may be treated appropriately for a diagnosis of early stage osteoarthritis. Osteoarthritis treatment goals are relieving pain, maintaining joint flexibility, and optimizing joint and overall function. Primary treatments include physical measures that involve rehabilitation; support devices; exercise for strength, flexibility, and endurance; patient education; and modifications in activities of daily living. Adjunctive therapies include drug treatment and surgery.

Moderate weight loss in patients with overweight often reduces pain and may even reduce progression of knee osteoarthritis. Rehabilitation techniques are best begun before disability develops. Exercises (range of motion, isometric, isotonic, isokinetic, postural, strengthening) maintain range of motion and increase the capacity for tendons and muscles to absorb stress during joint motion. Exercise can sometimes arrest or even reverse hip and knee osteoarthritis. Aquatic exercises are recommended because they spare the joints from stress. Stretching exercises should be done daily. In osteoarthritis of the spine, knee, or thumb carpometacarpal joint, various supports can relieve pain and increase function, but to preserve flexibility, they should be accompanied by specific exercise programs. For medial knee osteoarthritis, orthoses designed to reduce knee load are preferred to lateral wedge insoles, which have yielded equivocal outcomes. In erosive osteoarthritis, range-of-motion exercises done in warm water can help prevent contractures.

Drug therapy is an adjunct to the physical program. Acetaminophen in dosages of up to 1 g orally 4 times a day may relieve pain and is generally safe in the absence of hepatic disease or considerable alcohol intake. More potent analgesics, such as tramadol or rarely opioids, may be required; however, these medications can cause confusion in older patients and are generally avoided. Duloxetine, a serotonin norepinephrine reuptake inhibitor, may modestly reduce pain caused by osteoarthritis. Topical capsaicin has been helpful in relieving pain in superficial joints by disrupting pain transmission.

Nonsteroidal anti-inflammatory drugs (NSAIDs), including selective cyclooxygenase-2 (COX-2) inhibitors or coxibs, may be considered if patients have refractory pain or signs of inflammation (eg, redness, warmth). NSAIDs may be used simultaneously with other analgesics (eg, tramadol, rarely opioids) to provide better relief of symptoms. Topical NSAIDs may be of value for superficial joints, such as the hands and knees. Topical NSAIDs may be of particular value in older patients, because systemic NSAID exposure is reduced, minimizing risk of drug adverse effects. Gastric protection should be considered when using NSAIDs on a regular basis in older patients.

Muscle relaxants such as cyclobenzaprine, metaxalone, and methocarbamol (usually in low doses) occasionally relieve pain that arises from muscles strained by attempting to support osteoarthritis joints, yet strong evidence is lacking unless there is coexistent central sensitization. In older patients, however, they may cause more adverse effects than relief.

Hyaluronic acid formulations can be injected into the knee and provide some pain relief in some patients for prolonged periods of time. They should not be used more often than every 6 months. The treatment is a series of 1 to 5 weekly injections.

Glucosamine sulfate 1500 mg orally once/day has been suggested to relieve pain and slow joint deterioration; chondroitin sulfate 1200 mg once/day has also been suggested for pain relief.

Other adjunctive measures may reduce pain, including massage, heating pads, weight loss, acupuncture, and transcutaneous electrical nerve stimulation (TENS). Laminectomy, osteotomy, and total joint replacement should be considered if nonsurgical approaches fail.

Disease-modifying anti-rheumatic drugs (DMARDs) may improve articular pain and functional disability in OA patients. The most common conventional DMARDs are methotrexate, sulfasalazine, hydroxychloroquine, and leflunomide. Biological therapies include adalimumab, the IL-6R inhibitor tocilizumab, interleukin 1 inhibitors, etanercept, etc. Targeted synthetic DMARDs, include, for example, the JAK inhibitors baricitinib and tofacitinib

### Data Analysis

A signature pattern of altered immune cell populations can be generated from a biological sample using any convenient protocol, for example as described below. The readout can be a mean, average, median or the variance or other statistically or mathematically-derived value associated with the measurement. The marker readout information can be further refined by direct comparison with the corresponding reference or control pattern. A population distribution pattern can be evaluated on a number of points: to determine if there is a statistically significant change at any point in the data matrix relative to a reference value; whether the change is an increase or decrease in the population frequency; and the like. The absolute values obtained for each marker under identical conditions will display a variability that is inherent in live biological systems and also reflects the variability inherent between individuals.

Following obtainment of the signature pattern from the sample being assayed, the signature pattern can be compared with a reference or base line profile to make a prognosis regarding the phenotype of the patient from which the sample was obtained/derived.

In certain embodiments, the obtained signature pattern is compared to a single reference/control profile to obtain information regarding the phenotype of the patient being assayed. In yet other embodiments, the obtained signature pattern is compared to two or more different reference/control profiles to obtain more in depth information regarding the phenotype of the patient. For example, the obtained signature pattern can be compared to a positive and negative reference profile to obtain confirmed information regarding whether the patient has the phenotype of interest.

The data can be subjected to non-supervised hierarchical clustering to reveal relationships among profiles. For example, hierarchical clustering can be performed, where the Pearson correlation is employed as the clustering metric. One approach is to consider a patient disease dataset as a “learning sample” in a problem of “supervised learning”. CART is a standard in applications to medicine (Singer (1999) Recursive Partitioning in the Health Sciences, Springer), which can be modified by transforming any qualitative features to quantitative features; sorting them by attained significance levels, evaluated by sample reuse methods for Hotelling's T² statistic; and suitable application of the lasso method. Problems in prediction are turned into problems in regression without losing sight of prediction, indeed by making suitable use of the Gini criterion for classification in evaluating the quality of regressions.

Other methods of analysis that can be used include logistic regression. One method of logic regression Ruczinski (2003) Journal of Computational and Graphical Statistics 12:475-512. Logic regression resembles CART in that its classifier can be displayed as a binary tree. It is different in that each node has Boolean statements about features that are more general than the simple “and” statements produced by CART.

Another approach is that of nearest shrunken centroids (Tibshirani (2002) PNAS 99:6567-72). The technology is k-means-like, but has the advantage that by shrinking cluster centers, one automatically selects features (as in the lasso) so as to focus attention on small numbers of those that are informative. The approach is available as Prediction Analysis of Microarrays (PAM) software, a software “plug-in” for Microsoft Excel, and is widely used. Two further sets of algorithms are random forests (Breiman (2001) Machine Learning 45:5-32 and MART (Hastie (2001) The Elements of Statistical Learning, Springer). These two methods are already “committee methods.” Thus, they involve predictors that “vote” on outcome. Several of these methods are based on the “R” software, developed at Stanford University, which provides a statistical framework that is continuously being improved and updated in an ongoing basis.

Other statistical analysis approaches including principle components analysis, recursive partitioning, predictive algorithms, Bayesian networks, random forest, and neural networks.

The analysis and database storage can be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data can be used for a variety of purposes, such as patient monitoring, initial diagnosis, clinical trial analysis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer can be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language can be a compiled or interpreted language. Each such computer program is preferably stored on a storage media or device readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

### Kits

In some embodiments, the invention provides kits for the classification, diagnosis, prognosis, theragnosis, and/or prediction of early OA in a subject. The kit may further comprise a software package for data analysis of the cellular state and its physiological status, which may include reference profiles for comparison with the test profile and comparisons to other analyses as referred to above. The kit may also include instructions for use for any of the above applications.

Kits provided by the invention may comprise one or more of the affinity reagents described herein. A kit may also include other reagents that are useful in the invention, such as modulators, fixatives, containers, plates, buffers, therapeutic agents, instructions, and the like.

Kits provided by the invention can comprise one or more labeling elements. Non-limiting examples of labeling elements include small molecule fluorophores, proteinaceous fluorophores, radioisotopes, enzymes, antibodies, chemiluminescent molecules, biotin, streptavidin, digoxigenin, chromogenic dyes, luminescent dyes, phosphorous dyes, luciferase, magnetic particles, beta-galactosidase, amino groups, carboxy groups, maleimide groups, oxo groups and thiol groups, quantum dots, chelated or caged lanthanides, isotope tags, radiodense tags, electron-dense tags, radioactive isotopes, paramagnetic particles, agarose particles, mass tags, e-tags, nanoparticles, and vesicle tags.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Reports

In some embodiments, providing an evaluation of a subject for a classification, diagnosis, prognosis, theranosis, and/or prediction of early OA includes generating a written report that includes the artisan's assessment of the subject's state of health, including, for example, a “diagnosis assessment”, of the subject's prognosis, i.e. a “prognosis assessment”, and/or of possible treatment regimens, i.e. a “treatment assessment”. Thus, a subject method may further include a step of generating or outputting a report providing the results of an assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to a diagnosis assessment, a prognosis assessment, and/or a treatment assessment and its results. A subject report can be completely or partially electronically generated. A subject report includes at least a diagnosis assessment, and/or a suggested course of treatment to be followed. A subject report can further include one or more of: 1) information regarding the testing facility; 2) service provider information; 3) subject data; 4) sample data; 5) an assessment report, which can include various information including: a) test data, where test data can include an analysis of cellular signaling responses to activation, b) reference values employed, if any.

The report may include information about the testing facility, which information is relevant to the hospital, clinic, or laboratory in which sample gathering and/or data generation was conducted. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample gathering and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including subject medical history as well as administrative subject data (that is, data that are not essential to the diagnosis, prognosis, or treatment assessment) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the susceptibility prediction and, if different from the ordering physician, the name of a staff physician who is responsible for the subject's care (e.g., primary care physician).

The report may include a sample data section, which may provide information about the biological sample analyzed, such as the source of biological sample obtained from the subject (e.g. blood, type of tissue, etc.), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information generated after processing of the data as described herein. The interpretive report can include a prognosis of the likelihood that the patient will develop preeclampsia. The interpretive report can include, for example, results of the analysis, methods used to calculate the analysis, and interpretation, i.e. prognosis. The assessment portion of the report can optionally also include a Recommendation(s).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., a diagnosis, a prognosis, or a prediction of responsiveness to a therapy).

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

While the role of the immune system is implicated in OA pathogenesis, the details remain undefined. Here, we have identified OA-associated features in patients' peripheral blood immune cells using single-cell mass cytometry by time-of-flight (cyTOF). Comparison of healthy donors and OA patients' peripheral blood mononuclear cells (PBMC) revealed a significant expansion of CD24⁺CD27⁺IgD switched memory B cells and subsets of CD127⁺ memory T cells in OA. To test if this signature can be predictive of early OA, we applied supervised machine learning to immune profile data of patients with anterior cruciate ligament (ACL) tears and degenerative meniscal tears (DMT), two populations known to be at enhanced risk for OA. The OA-associated immune perturbations were selectively evident in many DMT and some ACL patients, demonstrating that a combination of mass cytometry and machine learning can be used for early OA prognosis and stratification of patients.

In this study, we have distinguished immune populations differentially abundant in the peripheral blood of OA patients using mass cytometry (cyTOF) and applied machine learning to identify OA-like immune features. We have additionally profiled peripheral blood of patients with anterior cruciate ligament (ACL) tears and degenerative meniscal tears (DMT), two populations known to be at enhanced risk for OA. This is the first study to comprehensively define an immune cell atlas for OA patients as well as for patients post ACL or DMT injuries.

### Results

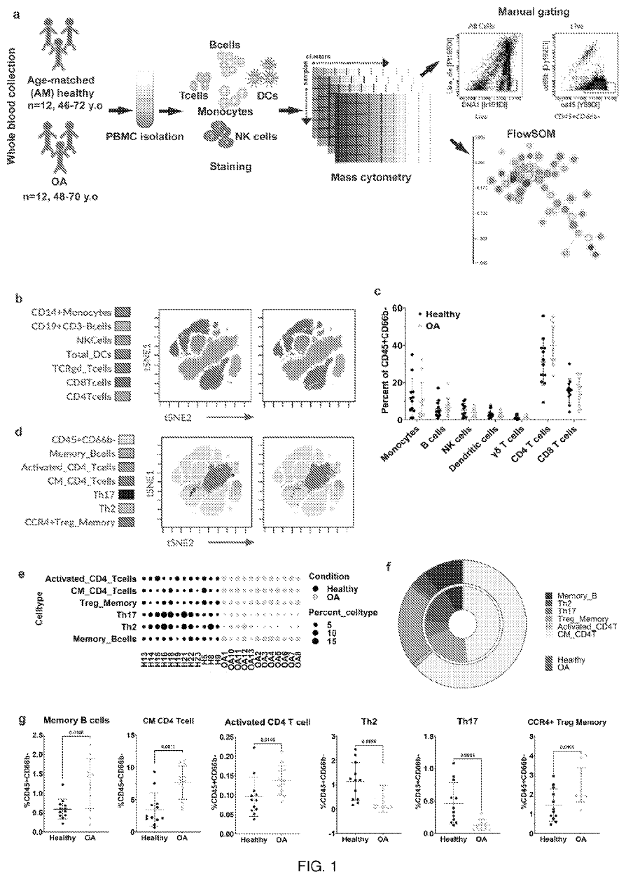

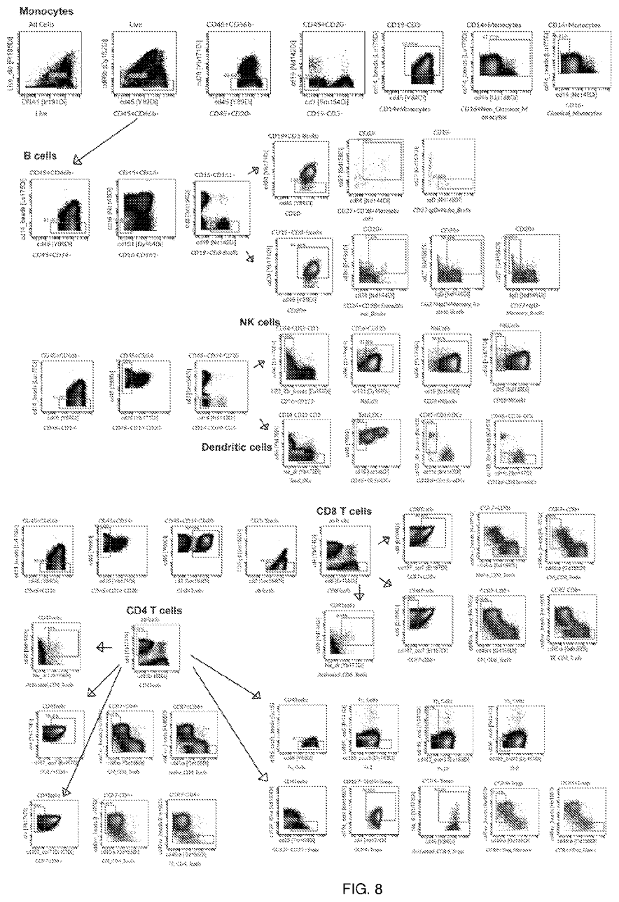

B and CD4 T cell subtypes show differential abundance between healthy and OA patients. Peripheral blood mononuclear cells (PBMC) isolated from age-matched OA patients with evident radiographic OA (n=12) and healthy individuals with no known history of OA (n=12) were stained with a 29-marker antibody panel (Fluidigm) to identify major immune cell populations by mass cytometry (FIG. 1a). A manual gating strategy was initially employed to identify defined immune cell populations that are differentially abundant between OA and healthy individuals (FIG. 8). Since the ages in the donor cohort covered a large span from 48 years to 72 years, we controlled for age-associated changes both by using age-matched controls as much as possible and through statistical correction for age (non-parametric Quade test) in our analyses. No statistically significant differences were noted between OA and healthy PBMC in the total frequencies of major immune populations namely, CD14⁺ monocytes, NK cells, dendritic cells, CD4T cells, γδT cells (TcRgd cells), CD8T cells, and B cells between healthy and OA cohorts (FIG. 1b-c). However, frequencies of memory B cells and CD4T cell subtypes namely, activated CD4T cells, central memory (CM), memory regulatory T cells (Treg), Th17 and Th2 were found to be differentially abundant in individual OA profiles compared to healthy profiles (FIG. 1e). The proportional distribution of the memory B cell and CD4T subpopulations were distinct (FIG. 1f), and the means were significantly different between OA and healthy cohorts (FIG. 1G). Specifically, memory B cells (CD20⁺CD27⁺IgD⁻), CM CD4T cells (CD4⁺CCR7⁺CD45RO⁺CD45RA⁻), activated CD4T cells (CD4⁺CD38⁺HLADR⁺) and memory Treg populations (CD25+CD127−CCR4⁺CD45RA⁻CD45RO⁺) were expanded whereas T helper subsets, Th17 (CD4⁺CXCR5⁻CXCR3⁻CCR6⁺) and Th2 (CD4⁺CXCR5⁻CXCR3⁻CCR6⁻), were depleted in OA PBMC.

Differential abundance of B cell repertoire in OA. A comprehensive analysis by way of unsupervised hierarchical clustering by FlowSOM performed on specific immune cell types identified distinct subpopulations that could not be distinguished by traditional markers used in manual gating. Distinct B cell clusters were identified to have different frequencies in OA compared to the age-matched healthy cohort PBMC (FIG. 2a). Based on the expression of CD27 and IgD, we distinguished naïve (CD20⁺CD27⁻IgD⁺) from switched memory (CD20⁺CD27⁺IgD⁻) B cells (FIG. 2b). To note, manual gating did not reveal significant differences in total naïve B cells, and congruently, not all FlowSOM clusters with naïve B phenotype were different in OA. While 9 naïve B cell clusters out of 28 were significantly different in OA, only cluster 14 that was marked by relatively high CCR7, CXCR5 and HLA-DR, (FIG. 2b) was found to be significantly expanded in OA (FIG. 2c). The rest of the 8 naïve B cell clusters were depleted in OA and could be differentiated based on the presence or absence of the receptors TCRgd, CD45RO, CD28, CXCR3, CXCR5, CCR4, and CCR6 (FIG. 2c).

Among the memory B cells, the metacluster 6 representing all clusters with CD27⁺IgD⁻ switched memory phenotype was significantly expanded in OA (FIG. 2d), consistent with the trend in manually gated total switched memory B population. However, not all clusters identified by FlowSOM that comprised the switched memory B metacluster 6 were similarly expanded. Clusters 9, 43, and 44 differentiated by the elevated expression of CD24 were significantly increased (FIG. 2b, e). In contrast, clusters 16, 30, and 49 which displayed lower levels of CD24 were depleted in OA. Thus, the increase in CD24⁺ cells in a subset of switched memory B cells was distinctive in OA.

Two CD14⁺ monocyte clusters were depleted in OA as compared to the healthy cohort (FIG. 2e). Specifically, classical monocyte subset (CD14^{low}CD16⁻, cluster2) and non-classical subset (CD14⁺CD16⁺, cluster3) were significantly diminished in OA (FIG. 2f). The non-classical monocyte cluster 3 was distinguishable from other clusters in its high expression of CD25.

Differential abundance of T cell repertoire in OA. FlowSOM analysis of the CD4T cell population identified differentially abundant clusters of naïve (CD4⁺CCR7⁺CD45RO⁻CD45RA⁺), central memory (CD4⁺CCR7⁺CD45RO⁺CD45RA⁻), and effector memory phenotypes (CD4⁺CCR7⁻CD45RO⁺CD45RA⁻) in OA patients when compared to age-matched healthy cohort (FIG. 3a). The central memory CD4T cell cluster 9, marked by higher expression of CXCR5, was depleted whereas cluster 29, marked by high expression of CCR4, CXCR3, and CD127 was expanded in OA (FIG. 3b-c). Naïve CD4T clusters 8, 61, 33, 54 and 70 were significantly depleted in OA (FIG. 3d). The naïve CD4T clusters 8 and 61 were differentiated from clusters 33, 54 and 70 in higher expression of CCR6, CCR4 and CXCR3 (FIG. 3b). While the frequencies of the effector memory CD4T clusters 6, 31 and 38 were significantly depleted, clusters 22 and 66 were significantly expanded in OA (FIG. 3e). Cluster 22 was defined by a higher expression of CD127 compared to others while cluster 66 specifically expressed CD24.

FlowSOM further revealed significant depletion of CD8T cell clusters 9, 16, and 20 (FIG. 3f, h-i) in OA as compared to the age-matched healthy cohort. Clusters 9 and 16 were identified as effector memory CD8T cells (CD8⁺CCR7⁻CD45RO⁺CD45RA⁻) wherein cluster 9 was distinguishable from cluster 16 by higher expression of CXCR5 receptor (FIG. 3g). Cluster 20 was identified to be terminal effector CD8T cell (CD8⁺CCR7⁻CD45RO⁻CD45RA⁺) with higher expression of CD123, a receptor for IL-3 (FIG. 3g).

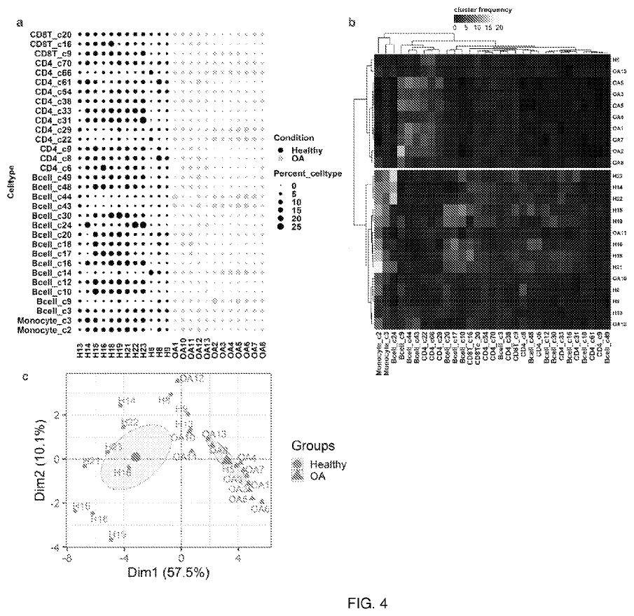

Overall immune profile stratifies OA and healthy phenotypes. Given the significant differences in the frequencies of clusters belonging to B cells, monocytes, CD4T, and CD8T cells between the healthy donors and OA patients, it was apparent that their overall immune profiles were distinct. Donor-to-donor differences were also apparent in both the healthy and OA cohorts (FIG. 4a). We, therefore, performed hierarchical clustering to discern the overall immune profiles for the healthy and OA cohort. Hierarchical clustering of the significantly different immune clusters, where distance was a measure of cosine similarity between clusters, revealed a clear stratification between the healthy and OA individuals (FIG. 4b). Interestingly, one healthy individual (H5) was an outlier and displayed an OA-like immune profile. These analyses also identified three OA patients (OA10, OA11, and OA12) that had a profile distinct from other OA patients and clustered with the healthy cohort (FIG. 4b). Another approach taken was a principal component analysis (PCA) on the significantly different immune clusters, which validated the stratification of the OA profiles into 2 different patient subsets distinct from the healthy cohort (FIG. 4c). OA1-8 and OA13 occupied a tight spatial cluster and were distinct from OA10, 11, and 12. The OA profile was associated with central memory CD4T cluster 9, effector memory CD4T clusters 22 and 66 along with memory B clusters 43, 44, 9, and the naïve B cluster14.

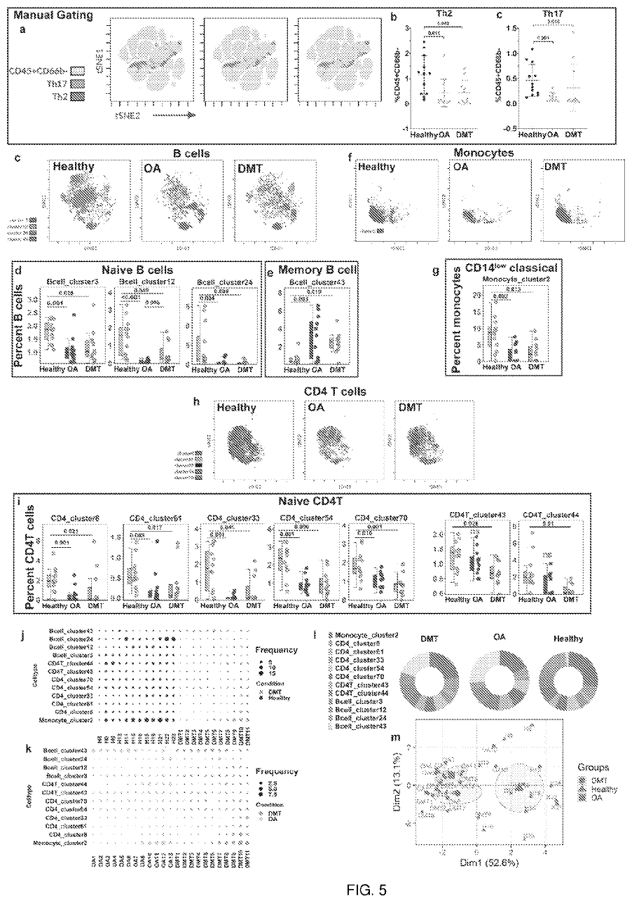

The immune landscape of Degenerative Meniscal Tear (DMT) patients resembles OA. Joint injuries have been implicated in increasing the risk of developing OA, with post-traumatic OA accounting for 12% of the overall burden of disease. Degenerative meniscal tears (DMT) are identified as one such injury-related risk factor of OA. To investigate whether correlations exist between the DMT and OA immune landscapes, we profiled PBMCs obtained from the blood of DMT patients before any surgical interventions were made (n=11) using the same 29-marker mass cytometry panel. All statistical measurements were conducted by controlling for age by non-parametric Quade test as before. Manual gating for defined immune populations revealed significant depletions in Th2 (p=0.043) and Th17 (p=0.018) subsets of CD4T cells in the DMT cohort when compared to the healthy cohort (FIG. 5a-b). While significant depletion of Th2 and Th17 cells were also observed in the OA cohort when compared to healthy (FIG. 1g, FIG. 5b), no significant difference was observed between DMT and OA groups (FIG. 5b).

To further discern OA-like perturbations in the immune cell abundance in the DMT cohort, we selected the FlowSOM clusters that were significantly different between healthy and OA as a framework to compare the frequencies of the same clusters in the DMT cohort. FlowSOM analyses revealed significant differences in the frequencies of immune clusters belonging to B cell, monocyte, and CD4T cell lineages in the DMT cohort when compared with healthy. Naïve B cell clusters 3, 12, and 24 in DMT overlapped with OA in being significantly depleted compared to healthy (FIG. 5c-d); similarly, memory B cell cluster 43 was significantly elevated in both DMT and OA cohorts (FIG. 5e). No significant difference in B cell cluster frequencies was observed between DMT and OA cohorts. For monocytes, the frequency of CD14^{low} classical monocyte cluster 2 was significantly depleted in DMT (p=0.013), as in OA (p=0.002) when compared to healthy (FIG. 5f-g).

No significant difference in the frequencies of CD8T cell clusters was noted among DMT, OA, and healthy cohorts. In the CD4T population, frequencies of naïve clusters (CCR7⁺CD45RA⁺CD45RO⁻) were significantly depleted in DMT when compared to healthy cohorts. All naïve CD4T clusters (8, 61, 33, 54, and 70) that were shown to be significantly depleted in OA (FIG. 3d), were also significantly depleted in DMT (FIG. 5h-i) when compared to the healthy cohort. The only difference observed between the DMT and OA immune landscape was in the naïve CD4T clusters 43 and 44, which were significantly depleted in DMT, but not in OA, when compared to the healthy cohort. Taken together, the data suggested that the majority of DMT immune features are remarkably shared with OA, specifically in the B cell, monocyte, and naïve CD4T cell subsets.

Overall, the frequency profile of the select clusters in DMT individuals was distinct from the healthy profile but similar to the OA profile (FIG. 5j-k)). Proportional distribution of the mean frequencies of the select clusters was similar between DMT and OA, and distinct from healthy (FIG. 5l). PCA analysis of the cluster frequencies in the 3 datasets displayed a closer association of the OA and DMT individuals overall in comparison to healthy, with a few outliers (FIG. 5m). Notably, the profiles of DMT10 and DMT11 overlapped with healthy cohort compared to the profiles of the rest of the DMT individuals demonstrating heterogeneity in the DMT profiles for OA-like immune features.

The immune landscape of ACL injury is different from the OA profile. Next, we examined the immune profiles of another high-risk group for developing PTOA i.e. patients with ACL injuries (n=11). PBMC from ACL patients were collected pre-surgery, under an approved IRB protocol, and their immune profiles generated by mass cytometry were compared with those of OA and healthy individuals. Statistical measurements for population differences were controlled for age effects by the Quade test, as described previously. No significant differences in the frequencies of defined immune populations were noted between ACL and healthy cohorts by manual gating.

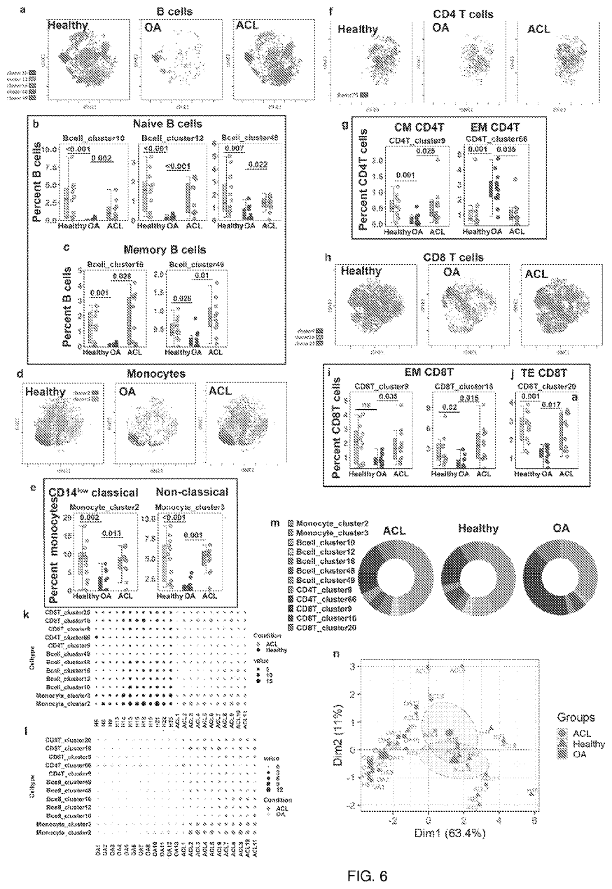

The framework of FlowSOM-identified immune clusters that were significantly different in OA compared to the healthy cohort was utilized to identify whether similar OA-like frequencies exist in the ACL immune profiles. Among the B cell clusters, naïve B clusters (10, 12, and 48) and switched memory B clusters (16 and 49) were significantly expanded in ACL when compared to OA (FIG. 6a-c). No significant difference was observed in the cluster frequencies between ACL and healthy cohorts. In monocyte clusters, no significant difference was noted between healthy and ACL cohorts, while both CD14 low classical cluster 2 and non-classical cluster 3 were significantly expanded in ACL and healthy cohorts when compared to the OA cohort (FIG. 6d-e). In the T cell repertoire, central memory CD4T cluster 9 and effector memory CD4T cluster 66 were significantly expanded in ACL and healthy when compared to OA cohort, whereas no significant difference was noted between ACL and healthy cohorts (FIG. 6f-g). Effector memory CD8T clusters (9 and 16) and terminal effector CD8T cell cluster 20 were significantly expanded in ACL and healthy cohorts when compared to OA (FIG. 6h-j). No significant difference in the CD8T cell cluster frequencies was observed between ACL and healthy populations. Thus, in contrast to the DMT population, the overall immune profile of the ACL population did not display OA-like features.

Using the immune clusters identified as differentially abundant between OA and healthy cohorts, it was apparent that in contrast to the DMT immune profile, the frequency distribution of immune clusters in ACL patients marked a closer resemblance with the healthy rather than the OA cohort (FIG. 6k-l). Overall proportional distribution of the select immune clusters in the ACL population was distinctly different from OA (FIG. 6m). In concordance with these data, PCA analyses demonstrated a substantial overlap between the confidence ellipses of ACL and healthy cohorts while being distinct from the confidence ellipse of the OA cohort. The only exception to this was the patient ACL1 that showed an overlap with the OA cohort (FIG. 6n).

A Machine learning algorithm to predict early OA. The prevalence of OA-like perturbations in the frequencies of select immune clusters in some at-risk patients (DMT and ACL) likely demonstrated signs of early OA pathogenesis. While invasive biopsies cannot be utilized in at-risk patients to assess disease progression, surveillance of the immune landscape in the blood can be an easy and routine methodology for the same. With this application in mind, we sought to apply machine learning methods to identify immune clusters (features) strongly associated with OA that can help identify an OA-like signature in the immune profiles of at-risk patients. Ultimately, we aimed to design a platform that can predict the probability of OA in individuals either known to be at-risk (with DMT or ACL injuries) or healthy with no known history of joint trauma.

The steps involved in feature selection and disease prediction in at-risk cohorts by machine learning are outlined in the schematic (FIG. 7a). As immune cell frequencies vary with age and given that DMT and ACL cohorts consisted of patients both above and below 40 years of age (outside of the age-matched cohort), we included an additional young healthy age group (age<40, n=11) to prevent the selection of age-associated features by the algorithm. Additionally, we pre-selected the FlowSOM-generated immune cluster frequencies that were found to be statistically significant between age-matched OA and healthy cohorts across all major immune cell types. This pre-selection was important to avoid irrelevant data, reduce complexity and minimize overfitting of the learning algorithm for accurate prediction. A ‘training cohort’ was then created to learn and select which immune clusters (features) were strongly associated with and were predictive of OA. This training cohort consisted of three groups-young healthy individuals (n=7;17-44 yrs old), older healthy individuals (n=12; 46-72 yrs old) age-matched to established OA patients (n=12; 48-70 yrs old). Boruta algorithm was used for the selection of important features which were then fed into a random forest algorithm to learn the ‘OA signature’ in the training dataset. Next, the algorithm was used to predict the OA signature in the ‘test cohort’ consisting of the at-risk groups, DMT (n=11) and ACL patients (n=11). As a proof-of-concept and to validate the accuracy of prediction by random forest algorithm, a small cohort of young healthy individuals (n=5), not included in the training dataset, was included in the test cohort, with a known outcome (i.e. healthy).

Only the immune cell clusters that were differentially abundant in the OA cohort, when compared with both the young and age-matched healthy cohorts, were pre-selected for analyses. These clusters included memory and naïve B cell clusters, effector memory, and naïve CD4T cell clusters (FIG. 7b-c). All clusters were significantly different between OA and age-matched healthy cohorts, while memory B cell cluster 9 and naïve B cell cluster 29 were significantly different between OA and young healthy cohorts (FIG. 7b). No significant difference in the frequencies of immune clusters between the age-matched and young healthy groups was noted. Boruta algorithm confirmed memory B cell clusters (3 and 9), naïve B cell clusters (19, 25, 26, and 36), effector memory CD4T cell clusters (50 and 69), and naïve CD4T cell cluster 65 to be important for predicting the OA signature (FIG. 7d). Switched memory B cell clusters 3 and 9 were identified as CD27⁺IgD⁻CD24⁺CD56^{low}, with differentially higher expression of CD24 in cluster 3 (FIG. 7e). Naïve B cell clusters 25 and 26 displayed CD27⁻IgD⁺CCR4⁺CXCR3⁺ phenotype, distinct from clusters 19 and 36 which were CD27⁻IgD⁺CCR4⁻CXCR3⁻ (FIG. 7e). Further, naïve B cell cluster 25 was marked by a higher expression of CD45RO while naïve B cell cluster 26 was distinct from others in the expression of CD56. Effector memory CD4T clusters 50 and 69 were identified by CCR7⁻CD45RA⁻CD45RO⁺ phenotype, with cluster 50 differing from cluster 69 in the expression of CD27 (FIG. 7f). Naïve CD4T cluster 65 had CCR7⁺CD45RO⁻CD45RA⁺CD27⁺ phenotype.

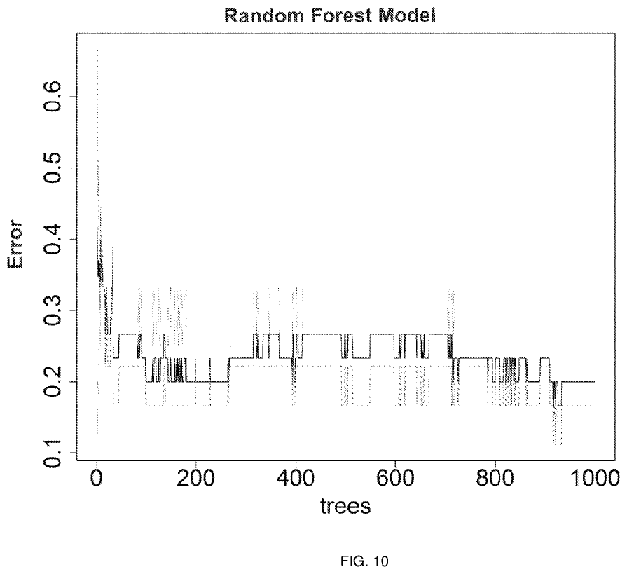

Based on the features selected by Boruta, the random forest classifier algorithm predicted the probability of disease state, either healthy or OA, in the test (DMT and ACL groups) and validation cohorts (new young healthy not included in training cohort). The accuracy of the optimal model used for prediction was 79.08% with an error rate of 20% (FIG. 10). As the error rate was high due to the small sample size, a probability of 70% or higher was considered acceptable for classifying a sample immune profile as predicted OA. The percent probabilities of heathy or OA states in the DMT, ACL, and young healthy validation cohorts as predicted by random forest model based on relevant features selected by Boruta are shown in FIG. 7g. The model correctly predicted all subjects in the validation cohort as healthy. In the DMT cohort, the model predicted five patient samples, DMT1-5 to have the OA signature. DMT1 and DMT2 had the highest probabilities (90.2% and 89.2%) of predicted OA, while DMT3, DMT4, and DMT5 had a slightly lower probability (>74%) of being predicted as OA. The rest of the DMT patients, DMT 6-11 were distinct from the other patients and were predicted to be healthy i.e. OA probability being lower than 60%. Even in this healthy group, DMT 6, 7, and 8 have a relatively higher probability of OA (˜60%) than DMT 9, 10 and 11 that had a very low probability of OA, demonstrating the range of the prediction algorithm. In the ACL cohort, the immune profile of only one patient sample, ACL1, was predicted to have an 87.4% chance of OA. All the other ACL patients were predicted to be healthy. In this group, while ACL3 and 4 showed a relatively higher (40 and 50%) probability of OA, the rest of the patients had a very low probability of OA (<30%). The OA patients profiled in this study were known to have radiographic OA as evident by KL scores of 2-3 in X-ray images. Upon closer inspection, while all other ACL patients showed a low KL score in the range of 0 to 1, ACL1 had a high KL score of 2. Even in this small cohort, this observation supports the prediction model. The KL scores of patients in the DMT cohort were, on the other hand, were not indistinguishable from one another, underscoring the need for alternative approaches like our platform for early OA prognosis.

Recent studies have elucidated the role of memory B cells in inflammation, pathogenesis, and relapse of various diseases, including RA prompting the exploration of B cell depletion approaches for therapeutic intervention in their pathogenesis. The role of B cells in OA pathogenesis is relatively unexplored. A remarkable feature that we observed in the OA cohort was a significant expansion of the switched memory B cell subpopulation (CD19⁺CD27⁺IgD⁻) in blood. Two distinct phenotypes of switched memory B cells were identified in OA peripheral blood that showed opposite trends—while CD27⁺IgD⁻CD24^{high} cells were expanded, CD27⁺IgD⁻CD24^{low} cells were found to be depleted. This is interesting as recent studies have reported the memory B CD27⁺IgD⁻CD24^{high} cells to be expanded in the RA blood, synovial fluid as well as synovium. It was suggested that these memory B CD27⁺IgD⁻CD24^{high} cells promote osteoclast formation and hence bone destruction in RA joints. Since the extent of cartilage and bone destruction in OA joints is slower and more distinct than in RA, it will be interesting to further explore the precise effect of these memory B CD27⁺IgD⁻CD24^{high} cells in OA. In addition to memory B cells, we also noted fewer frequencies of naïve B cell populations in the peripheral blood of OA patients. These observations highlight the power and significance of utilizing higher resolution techniques like mass cytometry since these differences could not be detected by previous analyses using flow cytometry with a limited number of cell-surface markers.

In the CD4T cell repertoire, there was a significant reduction in the frequencies of circulating Th2 and Th17 cells in the OA peripheral blood, whereas activated CD4T and CCR4+ memory Treg subsets were increased. Th17 cells have been reported to be detected at significant levels in the OA sera, synovial fluid, and membranes and appear to promote OA pathogenesis through the secretion of the pro-inflammatory cytokine IL-17. In contrast, the Th2 subset is believed to have a protective effect upon activation, through the anti-inflammatory cytokine IL-4. In a cohort of 40 OA patients, an accumulation of both Th17 and Th2 cells was observed in the synovial membranes and fluid, although the Th2 subset was smaller than Th17. A loss of IL-4-producing Th2 subset in OA peripheral blood is supported by a previous study. It is possible that an increased targeting and infiltration of the Th2 and Th17 CD4T cells in the OA joint leads to an increased abundance in the joint and a subsequent depletion from peripheral blood. Understanding the timing and role of the Th2/Th17 cells infiltration during OA pathogenesis and the underlying molecular pathways provides greater insight into the role of CD4T helper cells in OA.

Another hallmark of OA that was revealed by our analyses was the disruption in the balance of memory and naïve subsets of CD4T cells. While declining naïve and increasing memory T cell phenotypes are hallmarks of aging, there was a drastic reduction in naïve CD4 T clusters and a majority of memory CD4T clusters in the OA peripheral blood compared to age-matched healthy controls. Of note was a single subtype of central memory CD4T cells (cluster29) that was expanded in the OA cohort. This cluster was marked by high expression of CCR4, CXCR3, and CD127. Further, two subtypes of effector memory CD4T cells were also expanded in OA, one of which (cluster 22) was also defined by a higher expression of CD127. CD127 is a receptor for IL-7 and is essential for the long-term survival of memory T cells. As with CD4T cells, a reduction was also observed in memory CD8T clusters in OA compared to age-matched healthy controls. An imbalance in the T cell repertoire was therefore prominent in the OA landscape of immune cells.

OA patients are clinically defined by joint space narrowing detected by X-ray along with patient-reported outcomes on pain. Since these physical attributes of OA pathogenesis can be reached through multiple molecular routes including joint trauma, mechanical instabilities in gait, inflammation, metabolic disorders, and more, it is becoming increasingly clear that stratification of patients based on their molecular characteristics is required. Such a stratification would be helpful in risk identification as well as clinical treatments in patients, thereby taking the first steps toward a precision medicine approach. Hierarchical clustering of the OA cohort using the immune cell features identified 2 distinct OA profiles—9 OA patients had a profile that was distinct from the healthy cohort, while 3 other OA patients had a profile that was not. Thus, only a subset of OA patients may be driven by immune cell dysfunction. Our recent CyTOF study of OA cartilage also identified distinct patient subtypes based on inflammation states using a 20 patient cohort. Based on these observations, we report that immune cell profiling of peripheral blood can be greatly informative in patient stratification independently or in conjunction with clinical features like pain, BMI, and radiological imaging in a larger cohort of OA patients.

Another major bottleneck in OA treatment is the relatively late detection of the disease, which makes it difficult to reverse the pathogenesis and is the main reason for the lack of a disease-modifying OA drug in the clinic. Earlier detection of OA, before the irreversible clinical symptoms are apparent, would be a significant clinical breakthrough that will accelerate drug discovery efforts as well as help effective clinical trials for candidate drugs in early or moderate OA rather than late-stage OA. Although it takes many years for OA to manifest, it is known that patients with anterior cruciate ligament (ACL) tears or degenerative meniscal tears (DMT) are at a much higher risk for OA development. Novel methods in magnetic resonance imaging (MRI) have been applied for visualizing and quantifying changes in the cartilage before the advent of irreversible damages. However, high inter-patient variability in cartilage extracellular matrix composition may hinder the accuracy of predicting early OA based on MRI alone, especially in patients where initial matrix changes are not apparent. Our study, therefore, highlights the potential of utilizing patient-specific immune cell features either as a stand-alone or in combination with advanced MRI methods in the future to improve upon the current clinical methods for OA prognosis.

On profiling the immune cell landscape in these populations known to be at-risk for OA, we observed that there were significant similarities in the immune landscape of the OA cohort and some patients in the DMT cohort but not the ACL cohort. These analyses suggest that the immune cell features associated with OA might be present and detectable in the early stages of OA as well and can be utilized for early detection of the disease. We have therefore capitalized on the immune landscape in OA and at-risk patient groups to identify OA-like features by supervised machine learning. To this end, we have utilized the feature selection algorithm, Boruta, and predictive classification algorithm, Random Forest classifier, which have been utilized to identify disease-defining features and biomarkers in multiple studies. Before embarking on applying machine learning methodologies, we tested a preliminary predictive framework using PCA and hierarchical clustering. Utilizing significantly different immune FlowSOM clusters between age-matched OA and healthy cohorts (n=12) as relevant disease features, we built a predictive model by PCA. We used a dataset of OA and healthy patients (n=12 each) as the ‘training’ dataset, with the DMT and ACL patients (n=11 each) forming the ‘test’ datasets. A majority of DMT samples were predicted to align closely with the OA cohort in the PCA space generated by training data, while ACL samples were predicted to resemble the healthy cohort (FIG. 10). Congruent results were observed in the DMT and ACL datasets when hierarchical clustering with cosine dissimilarity as distance metric was performed to depict resemblance in immune profiles (FIG. 10). There were however clear outliers to the overall cohort trend, for example in the DMT cohort where DMT10 and DMT 11 overlapped with the healthy rather than the OA profile.

Our predictive model built with Boruta and random forest classifier algorithms validated the predictive PCA and clustering results, in addition to identifying the precise immune features capable of predicting OA phenotypic profile in at-risk cohorts. A combination of frequencies of switched memory B CD27⁺IgD⁻CD24^{high} phenotype, naïve B phenotype CD27⁻IgD⁺CXCR5⁺CD38⁺, effector memory CD4T with CD27^{low}CD127^{high}CCR6+ and CD27⁺CD127^{low}CCR6⁺, and naïve CD4T with CD27⁺CD127^{low}CXCR5⁺ phenotypes were identified as the predictive immune features for OA. The model correctly predicted all subjects in the validation cohort as healthy. While 5 out of 11 DMT patients were predicted to be OA-like, only 1 ACL patient was predicted to resemble the OA profile. The model, therefore, suggested ‘early OA’ in these 6 patients out of the 22 DMT and ACL patients that were screened. Importantly, the one ACL patient that was predicted to have early OA did show radiographic OA (KL score of 2) thereby supporting the prediction. X-ray images however did not discern between the DMT patients, underscoring the need for alternative diagnostic approaches.

To nullify age-related alterations in the abundance of the immune cells, our model includes a cohort of young healthy immune profiles with curated immune features in both training and test datasets. This combinatorial approach of rigorous statistical curation and supervised machine learning model with validation cohorts was essential to control for overfitting as well as effects of age. The accuracy of the optimal model used for prediction was 79.08% with an error rate of 20%. The error rate is high due to the small sample size, hence we used a probability of 70% or higher for predicting a sample immune profile as OA. Having a larger training dataset may reduce the error rate as well as increase the accuracy of the model. In addition, studies in patient-specific matched samples of blood and joint tissues will help to understand the link between immune cell abundance in circulation with local infiltration in the joint. Nonetheless, the data present the immune landscape in OA patients as well as the at-risk populations with ACL injuries and DMT. This study presents significant evidence that a tailored machine learning approach combined with single-cell immune cell data from peripheral blood can predict an early OA signature before the demonstration of clinical OA. It is, therefore, an important step forward in understanding OA-associated immunopathology for much-needed diagnostic, prognostic, and therapeutic approaches for the treatment of OA.

### Methods

Study design. The primary objective of the study was to profile immune cells in the peripheral blood of OA patients to highlight differences in their immune populations compared to healthy individuals. The next aim was to investigate the alignment of the immune landscape observed in OA to the immune profile of patients with degenerative meniscal tears (DMT) and anterior cruciate ligament (ACL) injuries, considered at-risk of developing OA post-injury. To this end, PBMCs were profiled by mass cytometry using a 29-marker mass cytometry panel (Human Immune Monitoring Panel Kit, Fluidigm). Immune profiles of age-matched cohorts of healthy (n=12) and OA (n=12) samples were first analyzed to identify differentially abundant populations. The immune profiles of DMT (n=11) and ACL (n=11) were matched to the OA profile to find similar perturbations in immune cell populations as observed in OA.

Peripheral blood collection and PBMC isolation. The whole blood of patients diagnosed with either OA, DMT or ACL injuries were collected by clinicians following written informed consent according to IRB protocols approved by Stanford University. The whole blood of healthy donors was procured from Stanford blood center. All blood samples were collected in EDTA vacutainer tubes and processed to isolate PBMCs within 6 hours of blood draw. PBMCs were isolated by Ficoll-based density gradient centrifugation. Briefly, whole blood was centrifuged at 200 g for 10 minutes at room temperature to remove plasma in the supernatant. Plasma-deficient blood was diluted with an equal volume of phosphate-buffered saline (PBS) free of Ca⁺⁺ and Mg⁺⁺ ions and layered over Ficoll-Paque Plus (density 1.077 g/mL, GE Healthcare). Density gradient centrifugation of blood layered over Ficoll was conducted at 1000 g for 15 minutes with minimum acceleration and zero deceleration settings at room temperature. The straw-colored buffy coat was carefully collected and treated with ACK lysing buffer (Gibco) to remove red blood cells. PBMCs were washed thrice with PBS, centrifuged, the PBMC pellet was frozen in FBS+20% DMSO and stored in liquid Nitrogen until staining for mass cytometry.

PBMC staining and mass cytometry. All staining and barcoding steps were performed using buffers, reagents, and protocols provided by Fluidigm. Briefly, frozen PBMCs were thawed in a 37° C. water bath, and approximately 3×10⁶ cells per sample were stained with 10 μM cisplatin to stain dead cells for 5 minutes at room temperature. Cisplatin was quenched using a serum-containing RPMI medium and washed in Maxpar cell staining buffer. PBMCs were fixed in Fix I buffer and permeabilized in 1× barcode perm buffer before barcoding using cell-Id 20-plex barcoding kit (Fluidigm) per manufacturer's instructions. Once barcoded, the PBMCs from all samples were combined into one tube and stained for 29 surface markers using metal-conjugated antibodies provided in the Maxpar human immune cell monitoring panel kit (catalog no. 210324, Fluidigm) following manufacturer's guidelines. Finally, stained PBMCs were labeled with cell-ID intercalator (dilution 1:1000, Fluidigm) to stain DNA. To normalize signal over runtime, the PBMCs were diluted in EQ four-element calibration beads (Fluidigm) diluted with water (1:10) before injection into cyTOF2 mass cytometer using the supersampler, housed at Shared FACS facility at Beckman Center, Stanford University. As only 20 samples could be barcoded and run at a time, 3 batches of barcoding were performed and run at different times in the same mass cytometer. One common sample was included in all three batches and runs to normalize batch effects.

Data cleaning, normalization, and de-barcoding. Data were acquired in fcs files. Signal normalization over runtime for each run was performed using Normalizer v0.3. All fcs files were concatenated and de-barcoded using ‘Premessa’ package in R software. Channel data was arcsine transformed and batch normalized across three runs with respect to the common sample using BatchAdjust( ) command-line application in R. Files were then uploaded into cloud-based platform Cytobank for gating and clustering and visualization.

Manual gating, hierarchical clustering and tSNE projections. CD45⁺CD66b⁻ cell population gated upon cisplatin—viable cells were classified for major immune populations and their subtypes using gating strategy provided by Fluidigm. Unsupervised hierarchical clustering by FlowSOM in Cytobank was used to identify subpopulations of major immune cell types from gated parent populations with optimized metacluster and cluster inputs namely: (a) CD45⁺CD66b⁻CD14⁻CD3⁺CD8⁻CD4⁺ population CD4T cell subpopulations (25 metaclusters and 81 clusters); (b) CD45⁺CD66b⁻CD14⁻CD3⁺CD4⁻CD8⁺ population CD8T cell subpopulations (11 metaclusters and 25 clusters); (c) CD45⁺CD66b⁻CD14⁻CD16⁻CD161⁻CD3⁻CD19⁺ population B cell subpopulations (10 metaclusters and 49 clusters); and (d) CD45⁺CD66b⁻CD20⁻CD19⁻CD3⁻CD14⁺ population monocyte subpopulations (5 metaclusters and 9 clusters). All events (cells) per sample file were utilized for FlowSOM analyses and abundances were represented as percent of total cells analyzed per sample. tSNE projections of manually gated populations and FlowSOM clusters were created using equal downsampled files in the Cytobank platform with perplexity=60 and theta=0.5.

Feature selection and disease prediction. For feature selection and random forest classification, the training dataset comprised of age-matched healthy (n=12) and OA (n=12) groups along with young healthy (n=7), whereas the test dataset consisted of DMT (n=11), ACL (n=11) and unused young healthy (n=5) groups. Statistically significant differences in FlowSOM clusters among all major immune cell types in the training dataset were compiled. Feature selection was performed using the Boruta package in R. Features (clusters) deemed important and not rejected Boruta were utilized for the disease prediction algorithm. Random forest classifier was performed for disease prediction (either OA or healthy) using the caret package in R. Tuning parameters for training the algorithm were 10-fold cross-validation repeated 10 times, 25 tunelength and 1000 number of trees. Young healthy data set was included in the training and test cohorts to validate that the algorithm can accurately predict healthy populations that are not age-matched with the OA cohort and thus eliminate age-associated perturbations in features. Percent probability of OA and healthy in test cohorts based on selected features were visualized by heatmap (‘Complexheatmap’ package in R).

Statistical methods for data analyses. Statistical differences between means of immune cell populations and cluster frequencies were first controlled for age-associated effects by non-parametric Quade test in both age-matched and non-matched datasets. Mann-Whitney test was used to measure significance when comparing two groups. When more than 2 groups were compared, pairwise comparisons were calculated by Dunnet's test for groups with unequal variances. Graphical data and heatmaps were created using R software.

Data availability: All data needed to evaluate the results are in the paper or in supplementary materials. Raw fcs files acquired from cytOF2 mass cytometer and subsequent gating and FlowSOM results are stored in Cytobank and will be shared upon request.

### REFERENCES

1. Felson, D. T. et al. Synovitis and the risk of knee osteoarthritis: the MOST Study. Osteoarthritis and Cartilage 24, 458-464 (2016).
2. Moradi, B. et al. Unicompartmental and bicompartmental knee osteoarthritis show different patterns of mononuclear cell infiltration and cytokine release in the affected joints: Mononuclear cells in knee OA. Clin Exp Immunol 180, 143-154 (2015).
3. Grandi, F. C. et al. Single-cell mass cytometry reveals cross-talk between inflammation-dampening and inflammation-amplifying cells in osteoarthritic cartilage. Sci. Adv. 6, eaay5352 (2020).
4. Croft, A. P. et al. Distinct fibroblast subsets drive inflammation and damage in arthritis. Nature 570, 246-251 (2019).
5. Fonseka, C. Y., Rao, D. A. & Raychaudhuri, S. Leveraging blood and tissue CD4+ T cell heterogeneity at the single cell level to identify mechanisms of disease in rheumatoid arthritis. Current Opinion in Immunology 49, 27-36 (2017).
6. Krieg, C. et al. High-dimensional single-cell analysis predicts response to anti-PD-1 immunotherapy. Nat Med 24, 144-153 (2018).
7. Good, Z. et al. Single-cell developmental classification of B cell precursor acute lymphoblastic leukemia at diagnosis reveals predictors of relapse. Nat Med 24, 474-483 (2018).
8. Brown, T. D., Johnston, R. C., Saltzman, C. L., Marsh, J. L. & Buckwalter, J. A. Posttraumatic Osteoarthritis: A First Estimate of Incidence, Prevalence, and Burden of Disease. Journal of Orthopaedic Trauma 20, 739-744 (2006).
9. Katz, J. N. & Martin, S. D. Meniscus-Friend or foe: Epidemiologic observations and surgical implications. Arthritis Rheum 60, 633-635 (2009).
10. Leandro, M. J., Cambridge, G., Ehrenstein, M. R. & Edwards, J. C. W. Reconstitution of peripheral blood B cells after depletion with rituximab in patients with rheumatoid arthritis. Arthritis Rheum 54, 613-620 (2006).
11. Kristyanto, H. et al. Persistently activated, proliferative memory autoreactive B cells promote inflammation in rheumatoid arthritis. Sci. Transl. Med. 12, eaaz5327 (2020).
12. Lee, D. S. W., Rojas, O. L. & Gommerman, J. L. B cell depletion therapies in autoimmune disease: advances and mechanistic insights. Nat Rev Drug Discov 20, 179-199 (2021).
13. Barr, T. A. et al. B cell depletion therapy ameliorates autoimmune disease through ablation of IL-6-producing B cells. Journal of Experimental Medicine 209, 1001-1010 (2012).
14. Nissimov, N. et al. B cells reappear less mature and more activated after their anti-CD20-mediated depletion in multiple sclerosis. Proc Natl Acad Sci USA 117, 25690-25699 (2020).
15. Souto-Carneiro, M. M. et al. Alterations in peripheral blood memory B cells in patients with active rheumatoid arthritis are dependent on the action of tumour necrosis factor. Arthritis Res Ther 11, R84 (2009).
16. Guo, X. et al. Accumulation of synovial fluid CD19+CD24hiCD27+ B cells was associated with bone destruction in rheumatoid arthritis. Sci Rep 10, 14386 (2020).
17. Ponchel, F. et al. Changes in peripheral blood immune cell composition in osteoarthritis. Osteoarthritis and Cartilage 23, 1870-1878 (2015).
18. Hussein, M. R. et al. Alterations of the CD4+, CD8+ T Cell Subsets, Interleukins-1β, IL-10, IL-17, Tumor Necrosis Factor-a and Soluble Intercellular Adhesion Molecule-1 in Rheumatoid Arthritis and Osteoarthritis: Preliminary Observations. Pathol. Oncol. Res. 14, 321-328 (2008).
19. Snelling, S. J. B. et al. Presence of IL-17 in synovial fluid identifies a potential inflammatory osteoarthritic phenotype. PLoS ONE 12, e0175109 (2017).
20. Mimpen, J. Y. et al. Interleukin-17 is a potential contributor to the inflammatory environment in the OA joint. Osteoarthritis and Cartilage 27, S379 (2019).
21. Rosshirt, N. et al. Proinflammatory T cell polarization is already present in patients with early knee osteoarthritis. Arthritis Res Ther 23, 37 (2021).
22. Deligne, C. et al. Differential expression of interleukin-17 and interleukin-22 in inflamed and non-inflamed synovium from osteoarthritis patients. Osteoarthritis and Cartilage 23, 1843-1852 (2015).
23. Ishii, H. et al. Characterization of infiltrating T cells and Th1/Th2-type cytokines in the synovium of patients with osteoarthritis. Osteoarthritis and Cartilage 10, 277-281 (2002).
24. Nikolich-Žugich, J. Ageing and life-long maintenance of T-cell subsets in the face of latent persistent infections. Nat Rev Immunol 8, 512-522 (2008).
25. Li, W. Q., Jiang, Q., Khaled, A. R., Keller, J. R. & Durum, S. K. Interleukin-7 Inactivates the Pro-apoptotic Protein Bad Promoting T Cell Survival. Journal of Biological Chemistry 279, 29160-29166 (2004).
26. Chetoui, N., Boisvert, M., Gendron, S. & Aoudjit, F. Interleukin-7 promotes the survival of human CD4⁺ effector/memory T cells by up-regulating Bcl-2 proteins and activating the JAK/STAT signalling pathway: IL-7 signalling in human effector/memory T cells. Immunology 130, 418-426 (2010).
27. Cinque, M. E., Dornan, G. J., Chahla, J., Moatshe, G. & LaPrade, R. F. High Rates of Osteoarthritis Develop After Anterior Cruciate Ligament Surgery: An Analysis of 4108 Patients. Am J Sports Med 46, 2011-2019 (2018).
28. Culvenor, A. G. et al. Prevalence of knee osteoarthritis features on magnetic resonance imaging in asymptomatic uninjured adults: a systematic review and meta-analysis. Br J Sports Med 53, 1268-1278 (2019).
29. Chu, C. R. et al. Visualizing pre-osteoarthritis: Integrating MRI UTE-T2* with mechanics and biology to combat osteoarthritis—The 2019 Elizabeth Winston Lanier Kappa Delta Award. J Orthop Res 39, 1585-1595 (2021).
30. Chu, C. R. et al. Quantitative Magnetic Resonance Imaging UTE-T2* Mapping of Cartilage and Meniscus Healing After Anatomic Anterior Cruciate Ligament Reconstruction. Am J Sports Med 42, 1847-1856 (2014).
31. Titchenal, M. R. et al. Cartilage Subsurface Changes to Magnetic Resonance Imaging UTE-T2* 2 Years After Anterior Cruciate Ligament Reconstruction Correlate With Walking Mechanics Associated With Knee Osteoarthritis. Am J Sports Med 46, 565-572 (2018).
32. Yang, C. et al. Identification of gene biomarkers in patients with postmenopausal osteoporosis. Mol Med Report (2018) doi:10.3892/mmr.2018.9752.
33. Wallentin, L. et al. Plasma proteins associated with cardiovascular death in patients with chronic coronary heart disease: A retrospective study. PLoS Med 18, e1003513 (2021).
34. Smith, B. P. et al. Identification of early liver toxicity gene biomarkers using comparative supervised machine learning. Sci Rep 10, 19128 (2020).

TABLE 1
Demographic information of samples.

| | Sample | | | | Sample | | |
|---|---|---|---|---|---|---|---|
| | designation | Age | Gender | | designation | Age | Gender |
| OA | OA1 | 59 | Male | Age-Matched Healthy | H5 | 63 | Female |
| | OA2 | 52 | Female | | H8 | 46 | Male |
| | OA3 | 65 | Male | | H9 | 55 | Male |
| | OA4 | 53 | Female | | H13 | 52 | Male |
| | OA5 | 48 | Female | | H14 | 47 | Female |
| | OA6 | 57 | Male | | H15 | 70 | Male |
| | OA7 | 70 | Unknown | | H16 | 68 | Female |
| | OA8 | 54 | Male | | H18 | 72 | Male |
| | OA10 | 60 | Female | | H19 | 63 | Male |
| | OA11 | 68 | Male | | H21 | 61 | Female |
| | OA12 | 48 | Female | | H22 | 54 | Male |
| | OA13 | 69 | Unknown | | H23 | 58 | Male |
| DMT | DMT1 | 47 | Male | Young Healthy | H1 | 33 | Male |
| | DMT2 | 55 | Female | | H2 | 42 | Female |
| | DMT3 | 30 | Male | | H3 | 30 | Female |
| | DMT4 | 69 | Male | | H4 | 44 | Female |
| | DMT5 | 48 | Male | | H6 | 37 | Male |
| | DMT6 | 59 | Male | | H7 | 32 | Female |
| | DMT7 | 60 | Male | | H10 | 33 | Female |
| | DMT8 | 54 | Male | | H11 | 37 | Male |
| | DMT9 | 47 | Male | | H12 | 44 | Female |
| | DMT10 | 38 | Male | | H17 | 17 | Female |
| | DMT11 | 25 | Male | | H20 | 30 | Male |
| ACL | ACL1 | 40 | Male | | | | |
| | ACL2 | 34 | Female | | | | |
| | ACL3 | 30 | Female | | | | |
| | ACL4 | 54 | Female | | | | |
| | ACL5 | 39 | Male | | | | |
| | ACL6 | 26 | Female | | | | |
| | ACL7 | 31 | Male | | | | |
| | ACL8 | 24 | Male | | | | |
| | ACL9 | 30 | Female | | | | |
| | ACL10 | 57 | Female | | | | |
| | ACL11 | 39 | Male | | | | |

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of determining the presence of early osteoarthritis (OA) in an individual, the method comprising
obtaining a blood sample from the individual;
performing single cell flow cytometry on the population of immune cells present in the blood for a plurality of markers;
clustering the single cells into discrete subpopulations based on the presence of at least a portion of the plurality of markers;
performing a predictive classification algorithm relative to a training data set to determine if the frequency of one or more predictive immune cell populations is indicative of the presence of early osteoarthritis.

2. The method of claim 1, wherein the predictive immune cell populations comprise one or more of: (1) switched memory B CD27⁺IgD⁻CD24^{high} cells, (2) naïve B CD27⁻IgD⁺CXCR5⁺CD38⁺ cells; effector memory CD4 T cells with (3) CD27^{low}CD127^{high}CCR6⁺ and (4) CD27⁺CD127^{low}CCR6⁺, and (5) naïve CD4 T cells with CD27⁺CD127^{low}CXCR5⁺ phenotype.

3. The method of claim 1 wherein the predictive immune cell populations comprise all of (1) switched memory B CD27⁺IgD⁻CD24^{high} cells, (2) naïve B CD27⁻IgD⁺CXCR5⁺CD38⁺ cells; effector memory CD4 T cells with (3) CD27^{low}CD127^{high}CCR6⁺ and (4) CD27⁺CD127^{low}CCR6⁺, and (5) naïve CD4 T cells with CD27⁺CD127^{low}CXCR5⁺ phenotype.

4. The method of claim 1, wherein the sample is physically contacted with a panel of affinity reagents specific for markers that distinguish subsets of immune cells.

5. The method of claim 1, wherein the plurality of markers comprises: CD27; IgD; CD24; CD38; CXCR5; and optionally CD19.

6. The method of claim 1, wherein the plurality of markers comprises: CD4; CD27; CD127; CCR6; and CXCR5.

7. The method of claim 1, wherein the plurality of markers comprises: CD19; CD4; CD24; CD27; CD38; CD17; CXCR5; CCR6; and IgD.

8. The method of claim 1, wherein the individual is a human individual with a condition or injury that can pre-dispose to OA.

9. The method of claim 1, wherein the individual is asymptomatic for OA.

10. The method of claim 1, wherein single cell flow cytometry comprises time-of-flight mass cytometry of at least 10¹ cells.

11. The method of claim 1, wherein classification utilizes a random forest algorithm trained on sample data from individuals with a condition predisposing to early OA.

12. The method of claim 1, wherein the clustering and predictive classification algorithm are analyzed by a computer processor comprising software configured for the purpose.

13. The method of claim 1, wherein the individual is treated in accordance with the classification.

14. The method of claim 1, wherein the individual is stratified for a clinical trial in accordance with the classification.

15. The method of claim 1, wherein a report of the classification is provided to the individual.
